(19) United States
(12) Patent Application Publication
(10) Pub. No.: US 2013/0338369 A1
(43) Pub. Date: Dec. 19, 2013

(54) Process for the Synthesis of Aminobiphenylene

(71) Applicants: Markus Heinrich, Langensendelbach (DE); Gerald Pratsch, Freising (DE)

(72) Inventors: Markus Heinrich, Langensendelbach (DE); Gerald Pratsch, Freising (DE)

(73) Assignee: BASF SE, Ludwigshafen (DE)

(21) Appl. No.: 14/003,399

(22) PCT Filed: Mar. 6, 2012
(86) PCT No.: PCT/EP12/53798

§ 371 Date: Sep. 5, 2013

(30) Foreign Application Priority Data
Mar. 7, 2011 (EP) 11001877.7
Jul. 15, 2011 (EP) 11005807.0

(51) Int. Cl. C07C 209/68; C07D 231/14; C07C 253/30; C07D 213/82; C07C 213/08

(52) U.S. Cl. CPC C07C 209/68; C07D 213/82; C07C 213/08; C07C 253/30; C07D 231/14
USPC 546/316; 564/442; 558/418; 548/374.1

(57) ABSTRACT

The present invention relates to a process for the synthesis of 2-aminobiphenylene and derivatives thereof by reacting a benzene diazonium salt with an aniline compound under basic reaction conditions.

The present invention relates to a process for synthesis of 2-aminobiphenyls and derivatives thereof by reaction of a benzenediazonium salt with an aniline compound under basic reaction conditions. This process is performable inexpensively and is based on selective reactions. Functionalized biphenyl compounds are of great interest especially as pharmaceuticals and crop protection agents, and as precursors of such active ingredients.

A wide range of organometallic methods is now available for mild and efficient synthesis of biaryl compounds.

The known organometallic methods, however, are also afflicted with disadvantages. The attractiveness thereof is reduced, for example, by high costs of the starting materials, especially in the case of palladium-catalyzed reactions, or inadequate environmental compatibility, as in the case of nickel. Catalytic processes using cobalt compounds and iron compounds have only been employable to a limited extent to date.

Simpler starting materials can be used when the biaryl coupling is effected via a CH bond activation on the aromatic system. In spite of numerous current studies in this field of research, the usable substrate spectrum is to date still very limited. Compared to the variety of organometallic transformations, which have been developed essentially in the last two decades, addition reactions of aryl radicals onto aromatic substrates are currently only rarely used.

It is a long time since the pioneering studies by Pschorr, Gomberg and Bachmann in the field of free-radical biaryl synthesis were conducted, in which aryldiazonium salts are traditionally used as free-radical precursors [M. Gomberg, W. E. Bachmann, J. Am. Chem. Soc. 1924, 46, 2339-2343, R. Pschorr, Chem. Ber. 1896, 29, 496-501]. However, a fundamental disadvantage of the intermolecular reaction regime is that the addition of aryl radicals onto commonly used substrates such as substituted benzenes is usually only slow, the result of which is that side reactions are promoted [J. C. Scaiano, L. C. Stewart, J. Am. Chem. Soc. 1983, 105, 3609-3614]. The success of free-radical biaryl syntheses is therefore frequently linked to specific conditions, with use of the substrate as the solvent [A. Nunez, A. Sanchez, C. Burgos, J. Alvarez-Builla, Tetrahedron 2004, 60, 6217-6224, P. T. F. McLoughlin, M. A. Clyne, F. Aldabbagh, Tetrahedron 2004, 60, 8065-8071] or intramolecular performance of the reaction [M. L. Bennasar, T. Roca, F. Ferrando, Tetrahedron Lett. 2004, 45, 5605-5609]. An improvement in the conventional Gomberg-Bachmann reaction has also been achieved by a reaction regime under phase transfer conditions [J. R. Beadle, S. H. Korzeniowski, D. E. Rosenberg, B. J. Garcia-Slanga, G. W. Gokel, J. Org. Chem. 1984, 49, 1594-1603].

It is clear from recently published review articles regarding free-radical biaryl synthesis that aryldiazonium salts are increasingly being replaced in current research by aryl chlorides, bromides and iodides as free-radical precursors [A. Studer, M. Brossart in Radicals in Organic Synthesis, Eds. P. Renaud, M. P. Sibi, 1st ed., Wiley-VCH, Weinheim, 2001, Vol. 2, 62-80; W. R. Bowman, J. M. D. Storey, Chem. Soc. Rev. 2007, 36, 1803-1822; J. Fossey, D. Lefort, J. Sorba, Free Radicals in Organic Chemistry, Wiley, Chichester, 1995, 167-180]. However, for generation of aryl radicals from aryl halides, it is usually necessary to use toxic organotin compounds or expensive organosilicon compounds. Recently, there have additionally been descriptions of organocatalytic biaryl syntheses [A. Studer, D. Curran, Angew. Chem. Int. Ed 2011, 50, 5018-5022], but these likewise require aryl bromides or iodides as starting materials.

Based on the fundamental attractiveness of aryldiazonium salts as precursors of aryl radicals [C. Galli, Chem. Rev. 1988, 88, 765-792], the reasons for which are particularly the low toxicity and easy obtainability from aniline compounds, it is a significant challenge to widen the known substrate spectrum with regard to the synthesis of biaryl compounds.

In this context, anilines in particular are an important substrate group.

The individual examples of addition reactions of aryl radicals on to aniline derivatives have long been known, but this synthesis method for biarylamines has gained no significance to date. Allan and Muzik [Chem. Abstr. 1953, 8705] report, for example, on the reaction of the diazonium salt of para-nitroaniline with benzidine and N,N,N′,N′-tetramethylbenzidine. In this case, only the tetramethyl derivative enters into the free-radical biaryl coupling, whereas the unsubstituted benzidine reacts by a non-free-radical mechanism to give the corresponding triazene. An effect similar to the methyl substitution can be achieved when aniline compounds are reacted not as free bases but as anilinium salts with aryl radicals. The first studies concerning the free-radical arylation of protonated 1,4-phenyldiamine were likewise described by Allan and Muzik [Z. J. Allan, F. Muzik, Chem. Listy 1954, 48, 52]. More recent studies under improved reaction conditions led to a significant widening of the substrate spectrum; however, the aniline compounds were always reacted in protonated form with the aryl radicals [Angew. Chem. Int. Ed. 2008, 47, 9130-9133, WO2010/000856, WO2010/037531]. Protonated anilines, however, are less reactive compared to unprotonated anilines. The resulting slow addition of the aryl radicals onto the protonated anilines leads to side reactions of the aryl radicals and, as a result of this, only to moderate yields of biarylamines. On the other hand, it is the prevailing opinion in connection with aryldiazonium salts that the protonation (or dialkyl substitution) of the amino group is essential, since only in this way is effective suppression of the otherwise prevalent reaction routes of triazene formation and azo coupling possible.

It was an object of the present invention to provide an improved process for preparing 2-aminobiphenyls and derivatives. For reasons of cost, and because of their easy obtainability, aryldiazonium salts should be used as aryl radical precursors.

Surprisingly, the object is achieved by a process based on the free-radical arylation of unprotonated aniline compounds.

Thus, the invention relates to a process for preparing a compound of the formula 3

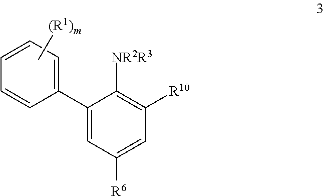

by reacting a compound of the formula 1

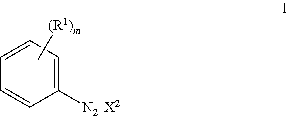

with a compound of the formula 2

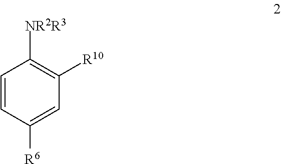

where
m is 0, 1, 2, 3, 4 or 5;
each R¹ is independently halogen, alkyl, haloalkyl, hydroxy, hydroxyalkyl, alkoxy, haloalkoxy, alkylthio, cycloalkyl, haloalkylthio, alkenyl, alkynyl, amino, nitro, cyano, —SO₃R⁵, —SO₂NH₂, —SO₂NHR⁴, —SO₂NR⁴R⁵, —COOR⁴, —CONHR⁴, —CONR⁴R⁵, —COR⁴, —OCOR⁴, —NR⁴R⁵, —NR⁴COR⁵, —NR⁴SO₂R⁵, alkylcarbonyl, haloalkylcarbonyl, alkenylcarbonyl, alkoxycarbonyl, haloalkoxycarbonyl, alkenyloxycarbonyl, alkylsulfonyl, haloalkylsulfonyl, alkylimino, aryl, aryloxy, arylcarbonyl, arylalkyl, heteroarylalkyl, arylalkoxycarbonyl, arylalkylimino or heteroaryl;
X⁻ is halide, hydrogensulfate, sulfate, tetrafluoroborate, acetate, trifluoroacetate, hexafluorophosphate, hexafluoroantimonate, the anion of an aromatic 1,2-dicarboximide or the anion of an aromatic 1,2-disulfonimide;
R² and R³ are each independently hydrogen, alkyl, hydroxyalkyl, aminoalkyl, cycloalkyl, haloalkyl, —(CH₂)ₙ—OR⁴, —(CH₂)ₙ—NR⁴R⁵, —(CH₂)ₙ—NR⁴COR⁵, —(CH₂)ₙ—NR⁴COOR⁵, —(CH₂)ₙ—COOR⁴, —(CH₂)ₙ—CONHR⁴, —(CH₂)ₙ—CONR⁴R⁵, —(CH₂)ₙ—SO₃R⁴, —(CH₂)ₙ—CN, arylalkyl, heteroarylalkyl, aryl or heteroaryl,
   or R² and R³ together form an alkylidene radical,
   or R² and R³ together with the nitrogen atom to which they are bonded form a nonaromatic 4-, 5-, 6- or 7-membered ring which may comprise 1, 2 or 3 further heteroatoms as ring members selected from O, S and N,
   or R² and R¹⁰ together with the atoms to which they are bonded form a nonaromatic 4-, 5-, 6- or 7-membered ring which may comprise 1, 2 or 3 further heteroatoms as ring members selected from O, S and N,
   or R³ and R¹⁰ together with the atoms to which they are bonded form a nonaromatic 4-, 5-, 6- or 7-membered ring which may comprise 1, 2 or 3 further heteroatoms as ring members selected from O, S and N;
n is in each case independently 1, 2, 3, 4, 5, 6, 7, 8, 9 or 10;
R⁴ is in each case independently hydrogen, alkyl, cycloalkyl, haloalkyl, arylalkyl, heteroarylalkyl, aryl or heteroaryl;
R⁵ is in each case independently hydrogen, alkyl, cycloalkyl, haloalkyl, arylalkyl, heteroarylalkyl, aryl or heteroaryl;
R⁶ is in each case independently hydrogen, halogen, alkyl, alkenyl, alkynyl, cycloalkyl, arylalkyl, heteroarylalkyl, haloalkyl, hydroxyalkyl, alkoxyalkyl, aryloxyalkyl, heteroaryloxyalkyl, aminoalkyl, —(CH₂)ₙ—NR⁴R⁵, —COOH, —CHO, —CN, —COR⁴, alkylcarbonyl, haloalkylcarbonyl, cycloalkylcarbonyl, arylalkylcarbonyl, alkenylcarbonyl, arylcarbonyl, heteroarylcarbonyl, —COOR⁴, alkoxycarbonyl, haloalkoxycarbonyl, cycloalkoxycarbonyl, arylalkoxycarbonyl, alkenyloxycarbonyl, aryloxycarbonyl, heteroaryloxycarbonyl, —CONHR⁴, —CONR⁴R⁵, amino, nitro, —NHR⁴, —NR⁴R⁵, 1-pyrrolidino, 1-piperidino, 1-morpholino, alkylimino, cycloalkylimino, haloalkylimino, arylalkylimino, —NR⁴COR⁵, —NR⁴COOR⁵, —NR⁴SO₂R⁵, hydroxyl, alkoxy, haloalkoxy, cycloalkoxy, arylalkyloxy, aryloxy, heteroaryloxy, —OCOR⁴, alkylcarbonyloxy, haloalkylcarbonyloxy, cycloalkylcarbonyloxy, arylalkylcarbonyloxy, arylcarbonyloxy, heteroarylcarbonyloxy, —OCONR⁴R⁵, —O—(CH₂)ₙ—OR⁴, —O—(CH₂)ₙ—NR⁴R⁵, —O—(CH₂)ₙ—NR⁴COR⁵, —O—(CH₂)ₙ—NR⁴COOR⁵, —O—(CH₂)ₙ—COOR⁴, —O—(CH₂)ₙ—CONHR⁴, —O—(CH₂)ₙ—CONR⁴R⁵, —O—(CH₂)ₙ—SO₃R⁴, —O—(CH₂)ₙ—SO₂R⁴, —O—(CH₂)ₙ—CN, —SH, alkylthio, haloalkylthio, cycloalkylthio, arylalkylthio, arylthio, heteroarylthio, alkylsulfonyl, haloalkylsulfonyl, cycloalkylsulfonyl, arylalkylsulfonyl, arylsulfonyl, heteroarylsulfonyl, —SO₂NH₂, —SO₂NHR⁴, —SO₂NR⁴R⁵, —SO₃R⁵, aryl or heteroaryl;
R¹⁰ is in each case independently hydrogen, halogen, alkyl, haloalkyl, hydroxyalkyl, cycloalkyl, arylalkyl, heteroarylalkyl, —(CH₂)_{q}—NR⁴R⁵, —(CH₂)_{q}—NR⁴COR⁵, —(CH₂)_{q}—NR⁴COOR⁵, —(CH₂)_{q}—COOR⁴, —(CH₂)_{q}—CONHR⁴, —(CH₂)_{q}—CONR⁴R⁵, —(CH₂)_{q}—SO₃R⁴, —(CH₂)_{q}—CN, aryl or heteroaryl;
q is in each case independently 1, 2, 3, 4, or 5,
   which comprises performing the reaction within the basic range.

In particular, a process for synthesis of optionally substituted 2-aminobiphenyls of the structure 3 by reaction of optionally substituted aryldiazonium salts of the structure 1 with optionally substituted aniline compounds of the structure 2 is described.

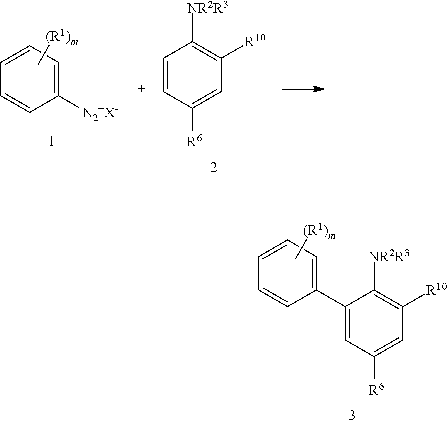

The intermediates formed here in the basic range are preferably compounds of the formulae 1a and/or 1b and/or 1c:

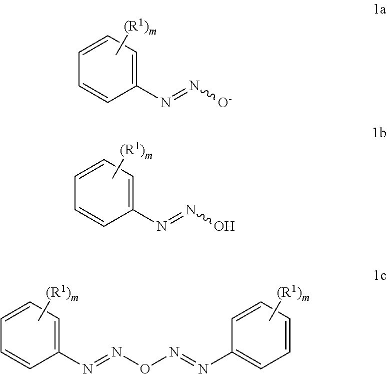

In these structures, the radicals are each as defined above. The counterion of compound 1a depends here on the base used. Preferred counterions are Na⁺ and K⁺.

The present invention thus further relates to a process for preparing a compound of the formula 3

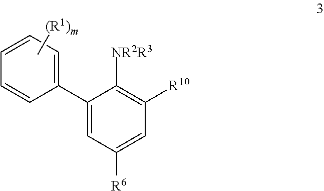

by, in a first step, converting a compound of the formula 1

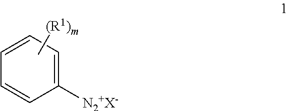

within the basic range to compounds of the formulae 1a and/or 1b and/or 1c

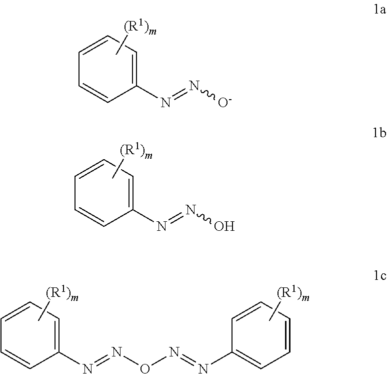

and, in a second step, reacting the compounds 1a and/or 1b and/or 1c within the basic range with a compound of the formula 2

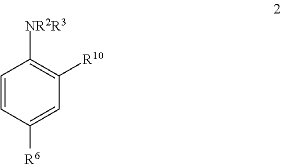

to give a compound of the formula 3

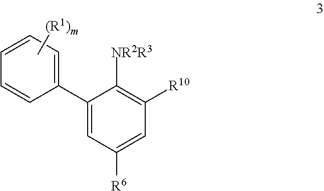

where all radicals are as defined above.

The compounds 1a, 1b and/or 1c react with release of nitrogen to give an aryl radical, which then reacts further with compound 2 to give compound 3.

The compounds of the structure 3 can be used, for example, as intermediates for preparation of biologically active compounds.

One example is the crop protection agent of the structure 5, which can be obtained by known processes from a compound of the structure 4 (US 2010/174094A1, WO 2006/024388A1, US 2008/269263A1, US 2010/069646A1). Organometallic syntheses of the compound of the structure 4, which, however, require much more expensive starting materials than the process according to the invention presented hereinafter, have likewise been described recently (WO2007/138089A1, US2010/185015A1).

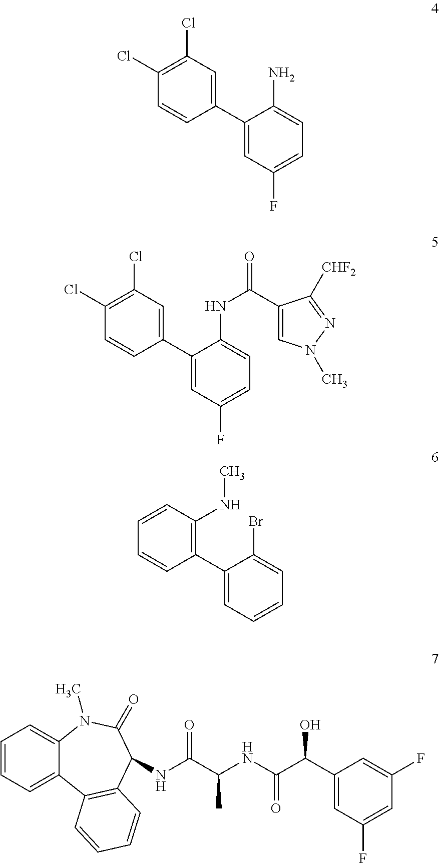

Proceeding from the compound of the structure 6, it is possible, for example, to prepare a γ-secretase inhibitor of the structure 7 (LY411575) (X. Pan, C. S. Wilcox, J. Org. Chem. 2010, 75, 6445-6451).

Additionally described is a process for synthesis of a compound of the formula 9, wherein, in a first step, a compound of the formula 1 is reacted with a compound of the formula 2 where R² and R³═H to give a compound of the formula 3 where R² and R³═H (preferably as described above), and, in a further step, the compound of the formula 3 is converted to a compound of the formula 8. The preparation of variously functionalized compounds of the formula 8 is possible under the conditions described in more detail below for diazotization of aromatic amines.

In a further step, the compounds of the formula 8 are converted by processes known from the literature to compounds of the formula 9. The reductive deamination to give 9 (R¹¹═H) can be performed under a wide variety of reaction conditions (for example in A. Wetzel, V. Ehrhardt, M. R. Heinrich, Angew. Chem. Int. Ed. 2008, 47, 9130-9133). Alternatively, halogen compounds, thiols, thioethers and nitriles of the formula 9 (R¹¹=—SH, —Salkyl, —Shaloalkyl, —Scycloalkyl, —S—(CH₂)_{q}-aryl, —S—(CH₂)_{q}-heteroaryl, —Saryl, —Sheteroaryl, halogen, cyano) are obtainable under the conditions of the Sandmeyer reaction (F. Minisci, F. Fontana, E. Vismara, Gazz. Chim. Ital. 1993, 123, 9-18). By insertion of SO₂ into the Sandmeyer reaction with copper halides, sulfonyl halides are obtained (R¹¹═SO₂Hal). Conversions of 8 under the conditions of aryldiazonium salt hydrolysis give hydroxyl-substituted compounds of the formula 9 (R¹¹═OH) (C. Galli, Chem. Rev. 1988, 88, 765-792). Nitro compounds and acid halides (R¹¹═NO₂ and CO-Hal respectively) are obtained as described in Galli (C. Galli, Chem. Rev. 1988, 88, 765-792). Conversions of 8 under the conditions of the Heck reaction give alkenyl-substituted compounds of the formula 9 (R¹¹=—CR¹⁴═CR¹⁵—COOH, —CR¹⁴═CR¹⁵—COOalkyl, —CR¹⁴═CR¹⁵—COOhaloalkyl, —CR¹⁴═CR¹⁵—CN, —CR¹⁴═CR¹⁵-aryl, —CR¹⁴═CR¹⁵-heteroaryl) (S. Sengupta, S. Bhattacharya, J. Chem. Soc. Perkin Trans 1, 1993, 17, 1943; A. Roglans, A. Pla-Quintana, M. Moreno-Manas, Chem. Rev. 2006, 106, 4622-4643). Reaction conditions for the reactions of compounds of the formula 8 with aromatic substrates to obtain compounds of the formula 9 (R¹¹=aryl, heteroaryl) are described many times in the introduction and in the remarks which follow.

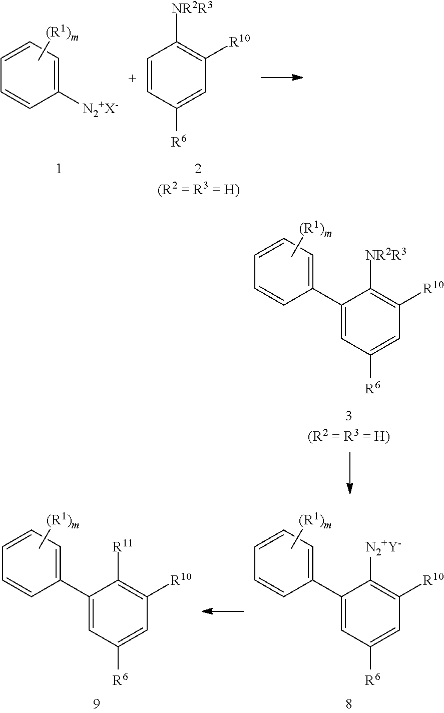

In this scheme:
R¹¹ is hydrogen, —OH, —SH, —Salkyl, —Shaloalkyl, —Scycloalkyl, —S—(CH₂)_{q}-aryl, —S—(CH₂)_{q}-heteroaryl, —Saryl, —Sheteroaryl, halogen, cyano, —CR¹⁴═CR¹⁵—COOH, —CR¹⁴═CR¹⁵—COOalkyl, —CR¹⁴═CR¹⁵—COOhaloalkyl, —CR¹⁴═CR¹⁵—CN, —CR¹⁴═CR¹⁵-aryl, —CR¹⁴═CR¹⁵-heteroaryl, —SO₂Hal, CO-Hal (Hal=halogen), NO₂, aryl or heteroaryl;
R¹⁴ is hydrogen, alkyl or haloalkyl;
R¹⁵ is hydrogen, alkyl, haloalkyl, —COOH, —COOalykl, —COOhaloalkyl, cyano, aryl, heteroaryl, —NHCOalkyl or —NHCOOalkyl;
Y⁻ is halide, hydrogensulfate, sulfate, tetrafluoroborate, acetate, trifluoroacetate, hexafluorophosphate, hexafluoroantimonate, the anion of an aromatic 1,2-dicarboximide or the anion of an aromatic 1,2-disulfonimide;
   and all further radicals are as defined above.

The present invention also relates to a process for preparing compounds 10

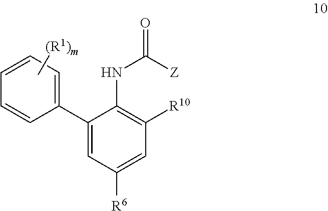

comprising the following step:
reacting a compound of the formula 1

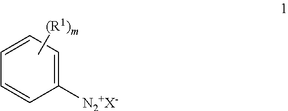

with a compound of the formula 2

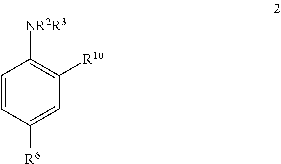

to give a compound of the formula 3

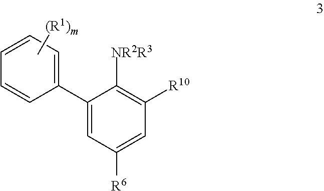

where R¹, R², R³, R⁶, R¹⁰, X⁻ and m are each as defined above; and
Z is aryl or 5- or 6-membered heteroaryl having 1, 2, 3 or 4 heteroatoms selected from N, O and S as ring members, where aryl and heteroaryl optionally bear 1, 2, 3 or 4 substituents selected from halogen, C₁-C₄-alkyl, C₁-C₄-haloalkyl, C₁-C₄-alkoxy and C₁-C₄-haloalkoxy;
   which comprises performing the reaction within the basic range.

In the context of the present invention, the terms used generically are defined as follows:

The prefix Cₓ-C_{y} in the respective case denotes the number of possible carbon atoms.

The term “halogen” in each case denotes fluorine, bromine, chlorine or iodine, specifically fluorine, chlorine or bromine, more preferably fluorine or chlorine.

The term “alkyl” denotes a linear or branched alkyl radical comprising 1 to 20 carbon atoms (C₁-C₂₀-alkyl), preferably 1 to 10 carbon atoms (C₁-C₁₀-alkyl), more preferably 1 to 6 carbon atoms (C₁-C₆-alkyl), particularly 1 to 4 carbon atoms (C₁-C₄-alkyl) and especially 1 to 3 carbon atoms (C₁-C₃-alkyl). Examples of C₁-C₃-alkyl are methyl, ethyl, propyl and 1-methylethyl (isopropyl). Examples of C₁-C₄-alkyl are, as well as those mentioned for C₁-C₃-alkyl, also n-butyl, 1-methylpropyl (sec-butyl), 2-methylpropyl (isobutyl) and 1,1-dimethylethyl (tert-butyl). Examples of C₁-C₆-alkyl are, as well as those mentioned for C₁-C₄-alkyl, also pentyl, hexyl and positional isomers thereof. Examples of C₁-C₁₀-alkyl are, as well as those mentioned for C₁-C₆-alkyl, also heptyl, octyl, 2-ethylhexyl, nonyl, decyl, 2-propylheptyl and positional isomers thereof. Examples of C₁-C₂₀-alkyl are, as well as those mentioned for C₁-C₁₀-alkyl, also undecyl, dodecyl, tridecyl, tetradecyl, pentadecyl, hexadecyl, heptadecyl, octadecyl, nonadecyl, docosyl and positional isomers thereof.

The term “haloalkyl”, as used herein and in the haloalkyl units of haloalkoxy, describes straight-chain or branched alkyl groups having 1 to 10 carbon atoms (C₁-C₁₀-haloalkyl), preferably 1 to 4 carbon atoms (C₁-C₄-haloalkyl) and especially 1 to 2 carbon atoms (C₁-C₂-haloalkyl), where some or all of the hydrogen atoms in these groups are replaced by halogen atoms. Examples of C₁-C₂-haloalkyl are chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromomethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl. Examples of C₁-C₄-haloalkyl are, as well as those mentioned for C₁-C₂-haloalkyl, also 3,3,3-trifluoroprop-1-yl, 1,1,1-trifluoroprop-2-yl, 3,3,3-trichloroprop-1-yl, heptafluoroisopropyl, 1-chlorobutyl, 2-chlorobutyl, 3-chlorobutyl, 4-chlorobutyl, 1-fluorobutyl, 2-fluorobutyl, 3-fluorobutyl, 4-fluorobutyl and the like. Preference is given to fluoromethyl, 2-fluoroethyl and trifluoromethyl.

The term “alkylidene” or “alkylene” denotes alkyl radicals which are bonded via a double bond and have 1 to 10 carbon atoms (C₁-C₁₀-alkylidene), preferably 1 to 4 carbon atoms (C₁-C₄-alkylidene) and especially 1 to 3 carbon atoms (C₁-C₂-alkylidene), where the alkyl radical optionally bears 1, 2 or 3 substituents selected from halogen, cycloalkyl, alkoxy and haloalkoxy. Examples are methylidene (═CH₂), ethylidene (═CHCH₃), 1-propylidene (═CHCH₂CH₃) or 2-propylidene [═C(CH₃)₂].

The term “cycloalkylidene” or “cycloalkylene” denotes cycloalkyl radicals which are bonded via a double bond and have 3 to 10 carbon atoms as ring members (C₃-C₁₀-cycloalkylidene), preferably 3 to 6 carbon atoms as ring members (C₃-C₆-cycloalkylidene), where the cycloalkyl radical optionally bears 1, 2 or 3 substituents selected from halogen, alkyl, haloalkyl, cycloalkyl, alkoxy, haloalkoxy. Examples are cyclopentylidene, cyclohexylidene or cycloheptylidene.

The term “haloalkylidene” or “haloalkylene” denotes haloalkyl radicals which are bonded via a double bond and have 1 to 10 carbon atoms (C₁-C₁₀-haloalkylidene), preferably 1 to 4 carbon atoms (C₁-C₄-haloalkylidene) and especially 1 to 3 carbon atoms (C₁-C₃-haloalkylidene). Examples are fluoromethylene (═CHF), 2-chloroethylidene (═CH—CH₂Cl) or 3-bromo-1-propylidene (═CH₂—CH₂—CH₂Br).

The term “arylalkylidene” or “arylalkylene” denotes aryl radicals bonded via an alkylidene unit, where the aryl group optionally bears 1, 2, 3, 4 or 5 substituents selected from halogen, alkyl, haloalkyl, alkoxy and haloalkoxy, and where the alkylidene unit preferably has 1 to 3 carbon atoms. Examples are benzylidene (═CH-phenyl), 1-naphthylidene (═CH-naphthyl) or ═CH—CH₂-phenyl.

The term “alkenyl” denotes a monounsaturated linear or branched aliphatic radical having 3 to 8 carbon atoms (C₃-C₈-alkenyl), preferably 3 or 4 carbon atoms (C₃-C₄-alkenyl). Examples thereof are propen-1-yl, propen-2-yl (allyl), but-1-en-1-yl, but-1-en-2-yl, but-1-en-3-yl, but-1-en-4-yl, but-2-en-1-yl, but-2-en-2-yl, but-2-en-4-yl, 2-methylprop-1-en-1-yl, 2-methylprop-2-en-1-yl and the like, preferably propenyl or but-1-en-4-yl.

The term “cycloalkyl” denotes a saturated alicyclic radical having 3 to 10 carbon atoms as ring members (C₃-C₁₀-cycloalkyl), preferably having 3 to 6 carbon atoms as ring members (C₃-C₆-cycloalkyl). Examples of C₃-C₆-cycloalkyl are cyclopropyl, cyclobutyl, cyclopentyl and cyclohexyl. Examples of C₃-C₁₀-cycloalkyl are, as well as those mentioned for C₃-C₆-cycloalkyl, cycloheptyl, cyclooctyl, cyclononyl and cyclodecyl. The cycloalkyl radicals may bear 1, 2 or 3 substituents selected from alkyl, alkoxy and halogen. Preference is given to cyclopropyl, cyclobutyl, cyclopentyl or cyclohexyl.

The term “alkoxy” denotes straight-chain or branched saturated alkyl groups bonded via an oxygen atom and comprising 1 to 10 carbon atoms (C₁-C₁₀-alkoxy), preferably 1 to 4 carbon atoms (C₁-C₄-alkoxy), where the alkyl radical optionally bears 1, 2 or 3 substituents selected from cycloalkyl, alkoxy and haloalkoxy. Examples of C₁-C₄-alkoxy are methoxy, ethoxy, n-propoxy, 1-methylethoxy (isopropoxy), n-butoxy, 1-methylpropoxy (sec-butoxy), 2-methylpropoxy (isobutoxy) and 1,1-dimethyloxy (tert-butoxy). Examples of C₁-C₁₀-alkoxy are, as well as those mentioned for C₁-C₄-alkoxy, pentyloxy, hexyloxy and the like. Preference is given to methoxy, ethoxy, n-propoxy and —OCH₂-cyclo-pentyl.

The term “haloalkoxy” describes straight-chain or branched saturated haloalkyl groups bonded via an oxygen atom and comprising 1 to 10 carbon atoms (C₁-C₁₀-haloalkoxy), preferably 1 to 4 carbon atoms (C₁-C₄-haloalkoxy). Examples thereof are chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, 1,1,2,2-tetrafluoroethoxy, 1-chloro-1,2,2-trifluoroethoxy, pentafluoroethoxy, 3,3,3-trifluoroprop-1-oxy, 1,1,1-trifluoroprop-2-oxy, 3,3,3-trichloroprop-1-oxy, 1-chlorobutoxy, 2-chlorobutoxy, 3-chlorobutoxy, 4-chlorobutoxy, 1-fluorobutoxy, 2-fluorobutoxy, 3-fluorobutoxy, 4-fluorobutoxy and the like; preference is given to fluoromethoxy, difluoromethoxy and trifluoromethoxy.

The term “cycloalkoxy” denotes a cycloalkyl radical bonded via an oxygen atom and having 3 to 10 carbon atoms as ring members (C₃-C₁₀-cycloalkoxy), preferably 3 to 6 carbon atoms as ring members (C₃-C₆-cycloalkoxy). Examples of C₃-C₆-cycloalkoxy are cyclopropyloxy, cyclobutyloxy, cyclopentyloxy and cyclohexyloxy. Examples of C₃-C₁₀-cycloalkoxy are, as well as those mentioned for C₃-C₆-cycloalkoxy, cycloheptyloxy, cyclooctyloxy, cyclononyloxy and cyclodecyloxy. The cycloalkyl radicals may bear 1, 2 or 3 substituents selected from alkyl and halogen. Preferred cycloalkoxy radicals are cyclopropyloxy, cyclobutyloxy, cyclopentyloxy and cyclohexyloxy.

The term “alkylcarbonyl” denotes alkyl radicals bonded via a carbonyl group and having 1 to 10 carbon atoms (C₁-C₁₀-alkylcarbonyl), where the alkyl radical optionally bears 1, 2 or 3 substituents selected from halogen, alkyl, haloalkyl, cycloalkyl, alkoxy and haloalkoxy. Examples thereof are methylcarbonyl (acetyl), ethylcarbonyl, propylcarbonyl, isopropylcarbonyl, n-butylcarbonyl, sec-butylcarbonyl, isobutylcarbonyl and tert-butylcarbonyl; preferably methylcarbonyl and ethylcarbonyl.

The term “haloalkylcarbonyl” denotes haloalkyl radicals bonded via a carbonyl group and having 1 to 10 carbon atoms (C₁-C₁₀-haloalkylcarbonyl). Examples thereof are fluoromethylcarbonyl, difluoromethylcarbonyl, trifluoromethylcarbonyl, 1-fluoroethylcarbonyl, 2-fluoroethylcarbonyl, 1,1-difluoroethylcarbonyl, 2,2-difluoroethylcarbonyl, 2,2,2-trifluoroethylcarbonyl, pentafluoroethylcarbonyl and the like; preference is given to fluoromethylcarbonyl, difluoromethylcarbonyl and trifluoromethylcarbonyl.

The term “alkylcarbonyloxy” denotes alkyl radicals bonded via a carbonyloxy group and having 1 to 10 carbon atoms (C₁-C₁₀-alkylcarbonyloxy), where the alkyl radical optionally bears 1, 2 or 3 substituents selected from cycloalkyl, alkoxy and haloalkoxy. Examples thereof are methylcarbonyloxy (acetoxy), ethylcarbonyloxy, propylcarbonyloxy and isopropylcarbonyloxy, preferably methylcarbonyloxy and ethylcarbonyloxy.

The term “haloalkylcarbonyloxy” denotes haloalkyl radicals bonded via a carbonyloxy group and having 1 to 10 carbon atoms (C₁-C₁₀-haloalkylcarbonyloxy), preferably 1 to 4 carbon atoms (C₁-C₄-haloalkylcarbonyloxy). Examples thereof are fluoromethylcarbonyloxy, difluoromethylcarbonyloxy, trifluoromethylcarbonyloxy, 1-fluoroethylcarbonyloxy, 2-fluoroethylcarbonyloxy, 1,1-difluoroethylcarbonyloxy, 2,2-difluoroethylcarbonyloxy, 2,2,2-trifluoroethylcarbonyloxy, pentafluoroethylcarbonyloxy and the like; preference is given to fluoromethylcarbonyloxy, difluoromethylcarbonyloxy and trifluoromethylcarbonyloxy.

The term “alkenylcarbonyl” denotes alkenyl radicals bonded via a carbonyl group and having 3 to 6 carbon atoms (C₃-C₆-alkenylcarbonyl), where the alkenyl radical optionally bears 1, 2 or 3 substituents selected from halogen, cycloalkyl, alkoxy and haloalkoxy. Examples thereof are propen-1-ylcarbonyl, propen-2-ylcarbonyl (allylcarbonyl), but-1-en-1-ylcarbonyl, but-1-en-2-ylcarbonyl, but-1-en-3-ylcarbonyl, but-1-en-4-ylcarbonyl, but-2-en-1-ylcarbonyl, but-2-en-2-ylcarbonyl, but-2-en-4-ylcarbonyl, 2-methylprop-1-en-1-ylcarbonyl, 2-methylprop-2-en-1-ylcarbonyl and the like; preference is given to propen-1-ylcarbonyl, propen-2-ylcarbonyl and but-1-en-4-ylcarbonyl.

The term “alkoxycarbonyl” denotes alkoxy radicals bonded via a carbonyl group and having 1 to 10 carbon atoms (C₁-C₁₀-alkoxycarbonyl), preferably 1 to 4 carbon atoms (C₁-C₄-alkoxycarbonyl), where the alkyl radical optionally bears 1, 2 or 3 substituents selected from cycloalkyl, alkoxy and haloalkoxy. Examples thereof are methoxycarbonyl, ethoxycarbonyl, propoxycarbonyl, isopropoxycarbonyl, n-butoxycarbonyl, sec-butoxycarbonyl, isobutoxycarbonyl and tert-butoxycarbonyl; preference is given to methoxycarbonyl, ethoxycarbonyl, propoxycarbonyl and isopropoxycarbonyl.

The term “haloalkoxycarbonyl” denotes haloalkoxy radicals bonded via a carbonyl group and having 1 to 10 carbon atoms (C₁-C₁₀-haloalkoxycarbonyl), preferably 1 to 4 carbon atoms (C₁-C₄-haloalkoxycarbonyl). Examples thereof are fluoromethoxycarbonyl, difluoromethoxycarbonyl, trifluoromethoxycarbonyl, 1-fluoroethoxycarbonyl, 2-fluoroethoxycarbonyl, 1,1-difluoroethoxycarbonyl, 2,2-difluoroethoxycarbonyl, 2,2,2-trifluoroethoxycarbonyl, pentafluoroethoxycarbonyl and the like; preference is given to fluoromethoxycarbonyl, difluoromethoxycarbonyl and trifluoromethoxycarbonyl.

The term “alkenyloxycarbonyl” denotes alkenyloxy radicals bonded via a carbonyl group and having 3 to 8 carbon atoms (C₃-C₈-alkenyloxycarbonyl), where the alkenyl radical optionally bears 1, 2 or 3 substituents selected from halogen, cycloalkyl, alkoxy and haloalkoxy. Examples thereof are allyloxycarbonyl and methallyloxycarbonyl, preferably allyloxycarbonyl.

The term “alkylsulfonyl” denotes alkyl radicals bonded via a sulfonyl group (SO₂) and having 1 to 10 carbon atoms (C₁-C₁₀-alkylsulfonyl), preferably 1 to 4 carbon atoms (C₁-C₄-alkylsulfonyl), where the alkyl radical optionally bears 1, 2 or 3 substituents selected from cycloalkyl, alkoxy and haloalkoxy. Examples thereof are methylsulfonyl, ethylsulfonyl, propylsulfonyl, isopropylsulfonyl, n-butylsulfonyl, sec-butylsulfonyl, isobutylsulfonyl and tert-butylsulfonyl; preferably methylsulfonyl, ethylsulfonyl, propylsulfonyl and isopropylsulfonyl.

The term “haloalkylsulfonyl” denotes haloalkyl radicals bonded via a sulfonyl group (SO₂) and having 1 to 10 carbon atoms (C₁-C₁₀-haloalkylsulfonyl), preferably 1 to 4 carbon atoms (C₁-C₄-haloalkylsulfonyl). Examples thereof are fluoromethylsulfonyl, difluoromethylsulfonyl, trifluoromethylsulfonyl, 1-fluoroethylsulfonyl, 2-fluoroethylsulfonyl, 1,1-difluoroethylsulfonyl, 2,2-difluoroethylsulfonyl, 2,2,2-trifluoroethylsulfonyl, pentafluoroethylsulfonyl and the like; preferably fluoromethylsulfonyl, difluoromethylsulfonyl and trifluoromethylsulfonyl.

The term “aryl”, as used herein and, for example, in the arylalkyl units of arylalkyl, denotes carbocyclic aromatic radicals having 6 to 14 carbon atoms, where the aryl group optionally bears 1, 2, 3, 4 or 5 substituents selected from halogen, cyano, nitro, alkyl, haloalkyl, alkoxy, alkoxycarbonyl or haloalkoxy. Examples thereof comprise phenyl, 4-chlorophenyl, 4-methoxyphenyl, naphthyl, fluorenyl, azulenyl, anthracenyl and phenanthrenyl. Preferably, aryl is phenyl or naphthyl, and especially phenyl.

The term “heteroaryl”, as used herein and, for example, in the heteroarylalkyl units of heteroarylalkyl, denotes aromatic radicals having 1 to 4 heteroatoms selected from O, N, S and SO₂, where the heteroaryl group optionally bears 1, 2, 3 or 4 substituents selected from halogen, nitro, cyano, alkyl, haloalkyl, alkoxy, alkoxycarbonyl or haloalkoxy. Examples thereof are 5- and 6-membered heteroaryl radicals having 1, 2, 3 or 4 heteroatoms selected from O, N, S and SO₂, such as pyrrolyl, 5-methyl-2-pyrrolyl, furanyl, 3-methyl-2-furanyl, thienyl, pyrazolyl, imidazolyl, oxazolyl, isoxazolyl, thiazolyl, isothiazolyl, triazolyl, tetrazolyl, pyridyl, pyrazinyl, pyridazinyl, pyrimidyl or triazinyl.

The term “arylcarbonyl” denotes aryl radicals bonded via a carbonyl group, where the aryl group optionally bears 1, 2, 3, 4 or 5 substituents selected from halogen, alkyl, haloalkyl, alkoxy or haloalkoxy. Examples thereof are phenylcarbonyl, 4-nitrophenylcarbonyl, 2-methoxyphenylcarbonyl, 4-chlorophenylcarbonyl, 2,4-dichlorophenylcarbonyl, 4-nitrophenylcarbonyl or naphthylcarbonyl, preferably phenylcarbonyl.

The term “arylalkyl” denotes aryl radicals bonded via an alkyl group, preferably a C₁-C₄-alkyl group (aryl-C₁-C₄-alkyl), especially a C₁-C₂-alkyl group (aryl-C₁-C₂-alkyl), where the alkyl radical optionally bears 1, 2 or 3 substituents selected from halogen, cycloalkyl, alkoxy and haloalkoxy, and where the aryl group optionally bears 1, 2, 3, 4 or 5 substituents selected from halogen, alkyl, haloalkyl, alkoxy and haloalkoxy. Examples thereof are 4-methoxybenzyl, benzyl, 2-phenylethyl (phenethyl) and the like; preferably benzyl and phenethyl.

The term “alkylimino” denotes a radical of the formula —N═R bonded via nitrogen, in which R is alkylene such as ═CH₂, ═CHCH₃, ═CHCH₂CH₃, ═C(CH₃)₂, ═CHCH₂CH₂CH₃, ═C(CH₃)CH₂CH₃ or ═CHCH(CH₃)₂. The alkylene radical of “alkylimino” optionally bears 1, 2 or 3 substituents selected from halogen, alkyl, haloalkyl, cycloalkyl, alkoxy, haloalkoxy.

The term “arylalkylimino” denotes a radical of the formula —N═R bonded via nitrogen, in which R is arylalkylene such as benzylidene (R═CH-phenyl). The aryl group in “arylalkylimino” may optionally bear 1, 2, 3, 4 or 5 substituents selected from halogen, alkyl, haloalkyl, alkoxy and haloalkoxy.

The term “hydroxyalkyl” denotes an alkyl group bearing one hydroxyl group, where the alkyl group optionally bears 1, 2 or 3 further substituents selected from halogen, alkyl, haloalkyl, cycloalkyl, alkoxy and haloalkoxy. Examples are —CH₂OH, —(CH₂)₂OH or —(CH₂)₃OH.

The term “alkynyl” denotes a linear or branched aliphatic radical with diunsaturation in the form of a carbon-carbon triple bond and having 3 to 8 carbon atoms (C₃-C₈-alkynyl). Examples thereof are propyn-3-yl, but-1-in-1-yl, but-1-in-3-yl, but-1-in-4-yl, but-2-in-1-yl, but-2-in-4-yl and the like; preferably propyn-3-yl and but-1-in-4-yl.

The term “heteroarylalkyl” denotes heteroaryl radicals bonded via an alkyl group, preferably a C₁-C₄-alkyl group (aryl-C₁-C₄-alkyl), especially a C₁-C₂-alkyl group (aryl-C₁-C₂-alkyl), where the alkyl radical optionally bears 1, 2 or 3 substituents selected from halogen, cycloalkyl, alkoxy, haloalkoxy, and where the heteroaryl group optionally bears 1, 2, 3 or 4 substituents selected from halogen, alkyl, haloalkyl, alkoxy and haloalkoxy. Examples thereof are 4-pyridylmethyl, 1-(4-pyridyl)ethyl, 2-(4-pyridyl)ethyl, 2-furanylmethyl, 1-(2-furanyl)ethyl, 2-(2-furanyl)ethyl and the like; preference is given to 4-pyridylmethyl.

The term “alkoxyalkyl” denotes alkoxy radicals which are bonded via an alkyl group having 1 to 4 carbon atoms and have 1 to 10 carbon atoms (C₁-C₁₀-alkoxy-C₁-C₄-alkyl), where the alkyl and/or alkoxy radicals optionally bear 1, 2 or 3 substituents selected from halogen and cycloalkyl. Examples thereof are methoxymethyl, ethoxymethyl, n-propoxymethyl, isopropoxymethyl, n-butoxymethyl, sec-butoxymethyl, isobutoxymethyl, tert-butoxymethyl, methoxyethyl, ethoxyethyl, propoxyethyl, isopropoxyethyl, n-butoxyethyl, sec-butoxyethyl, isobutoxyethyl, tert-butoxyethyl and the like; preference is given to methoxymethyl, ethoxymethyl, n-propoxymethyl, isopropoxymethyl, methoxyethyl, ethoxyethyl, propoxyethyl and isopropoxyethyl.

The term “aryloxyalkyl” denotes aryloxy radicals bonded via an alkyl group, preferably a C₁-C₄-alkyl group (aryl-C₁-C₄-alkyl), especially a C₁-C₂-alkyl group (aryl-C₁-C₂-alkyl), and having 6 to 14 carbon atoms, where the alkyl radical optionally bears 1, 2 or 3 substituents selected from halogen, cycloalkyl, alkoxy and haloalkoxy. The aryl group in “aryloxyalkyl” optionally bears 1, 2, 3, 4 or 5 substituents selected from halogen, alkyl, haloalkyl, alkoxy and haloalkoxy. Examples thereof are phenoxymethyl, phenoxyethyl, phenoxypropyl, phenoxybutyl, 1-naphthyloxymethyl, 1-(1-naphthyloxy)ethyl, 2-(1-naphthyloxy)ethyl, 1-(1-naphthyloxy)propyl, 2-(1-naphthyloxy)propyl, 3-(1-naphthyloxy)propyl and the like; preference is given to phenoxymethyl and phenoxyethyl.

The term “heteroaryloxyalkyl” denotes heteroaryloxy radicals bonded via an alkyl group, preferably a C₁-C₄-alkyl group (aryl-C₁-C₄-alkyl), especially a C₁-C₂-alkyl group (aryl-C₁-C₂-alkyl), and having 1 to 4 heteroatoms selected from O, N, S and SO₂, where the alkyl radical optionally bears 1, 2 or 3 substituents selected from halogen, cycloalkyl, alkoxy and haloalkoxy. The heteroaryl group in “heteroaryloxyalkyl” optionally bears 1, 2, 3 or 4 substituents selected from halogen, alkyl, haloalkyl, alkoxy and haloalkoxy. Examples thereof are 4-pyridyloxymethyl, 1-(4-pyridyloxy)ethyl, 2-(4-pyridyloxy)ethyl, 1-(4-pyridyloxy)propyl, 2-(4-pyridyloxy)propyl, 3-(4-pyridyloxy)propyl, 2-furanyloxymethyl, 1-(2-furanyloxy)ethyl, 2-(2-furanyloxy)ethyl, 1-(2-furanyloxy)propyl, 2-(2-furanyloxy)propyl, 3-(2-furanyloxy)propyl and the like; preference is given to 4-pyridyloxymethyl and 1-(4-pyridyloxy)ethyl.

The term “aminoalkyl” denotes an —NH₂ radical bonded via an alkyl group, where the alkyl group optionally bears 1, 2 or 3 substituents selected from halogen, alkyl, haloalkyl, cycloalkyl, alkoxy, haloalkoxy. Examples thereof are aminomethyl [—CH₂NH₂], aminoethyl [—(CH₂)₂NH₂] and the like, preference being given to —CH₂NH₂, —(CH₂)₂NH₂ or —(CH₂)₃NH₂.

The term “alkylaminoalkyl” denotes an —NHR⁴ or —NR⁴R⁵ radical bonded via an alkyl group, where R⁴ and R⁵ are each as defined above and the alkyl group optionally bears 1, 2 or 3 substituents selected from halogen, cycloalkyl, alkoxy and haloalkoxy. Examples are methylaminomethyl [—CH₂—NH—CH₃], N,N-dimethylaminomethyl [—CH₂—N(CH₃)₂], N,N-dimethylaminoethyl [—(CH₂)₂—N(CH₃)₂] and the like, preferably —CH₂—N(CH₃)₂ and —(CH₂)₂—N(CH₃)₂.

The term “cycloalkylcarbonyl” denotes cycloalkyl radicals bonded via a carbonyl group and having 3 to 10 carbon atoms (C₃-C₁₀-cycloalkylcarbonyl), preferably 3 to 6 carbon atoms (C₃-C₆-cycloalkylcarbonyl), as ring members, where the cycloalkyl radical optionally bears 1, 2 or 3 substituents selected from halogen, alkyl, haloalkyl, cycloalkyl, alkoxy and haloalkoxy. Examples thereof are cyclopropylcarbonyl, cyclobutylcarbonyl, cyclopentylcarbonyl, cyclohexylcarbonyl and the like; preferably cyclopropylcarbonyl, cyclopentylcarbonyl and cyclohexylcarbonyl.

The term “arylalkylcarbonyl” denotes arylalkyl radicals bonded via a carbonyl group, where the alkyl radical optionally bears 1, 2 or 3 substituents selected from halogen, cycloalkyl, alkoxy and haloalkoxy, and where the aryl group optionally bears 1, 2, 3, 4 or 5 substituents selected from halogen, alkyl, haloalkyl, alkoxy and haloalkoxy. Examples thereof are benzylcarbonyl, 2-phenylethylcarbonyl and the like; preferably benzylcarbonyl and 2-phenylethylcarbonyl.

The term “heteroarylalkylcarbonyl” denotes heteroarylalkyl radicals bonded via a carbonyl group and having 1 to 4 heteroatoms selected from O, N, S and SO₂, where the alkyl radical optionally bears 1, 2 or 3 substituents selected from halogen, cycloalkyl, alkoxy and haloalkoxy, and where the heteroaryl group optionally bears 1, 2, 3 or 4 substituents selected from halogen, alkyl, haloalkyl, alkoxy and haloalkoxy.

Examples thereof are 4-pyridylmethylcarbonyl, 1-(4-pyridyl)ethylcarbonyl, 2-furanylmethylcarbonyl, 1-(2-furanyl)ethylcarbonyl and the like; preference is given to 4-pyridylmethylcarbonyl.

The term “cycloalkoxycarbonyl” denotes cycloalkoxy radicals bonded via a carbonyl group and having 3 to 10 carbon atoms (C₃-C₁₀-cycloalkoxycarbonyl), preferably 3 to 6 carbon atoms (C₃-C₆-cycloalkoxycarbonyl), as ring members, where the cycloalkyl radical optionally bears 1, 2 or 3 substituents selected from halogen, alkyl, haloalkyl, cycloalkyl, alkoxy and haloalkoxy. Examples thereof are cyclopropyloxycarbonyl, cyclobutyloxycarbonyl, cyclopentyloxycarbonyl, cyclohexyloxycarbonyl, cycloheptyloxycarbonyl, cyclooctyloxycarbonyl, cyclononyloxycarbonyl and the like; preference is given to cyclopropyloxycarbonyl, cyclopentyloxycarbonyl and cyclohexyloxycarbonyl.

The term “arylalkoxycarbonyl” denotes arylalkoxy radicals bonded via a carbonyl group and having 6 to 14 carbon atoms, where the alkoxy radical optionally bears 1, 2 or 3 substituents selected from halogen, cycloalkyl, alkoxy and haloalkoxy, and where the aryl group optionally bears 1, 2, 3 or 4 substituents selected from halogen, alkyl, haloalkyl, alkoxy and haloalkoxy. Examples thereof are benzyloxycarbonyl, 2-phenylethyloxycarbonyl and the like; preference is given to benzyloxycarbonyl.

The term “aryloxy” denotes an aryl radical bonded via an oxygen atom, where the aryl group optionally bears 1, 2, 3, 4 or 5 substituents selected from halogen, alkyl, haloalkyl, alkoxy and haloalkoxy. Examples thereof are phenyloxy (phenoxy), naphthyloxy, fluorenyloxy and the like; preference is given to phenoxy.

The term “aryloxycarbonyl” denotes aryloxy radicals bonded via a carbonyl group and having 6 to 14 carbon atoms, where the aryl group optionally bears 1, 2, 3 or 4 substituents selected from halogen, alkyl, haloalkyl, alkoxy and haloalkoxy. Examples thereof are phenyloxycarbonyl (phenoxycarbonyl), naphthyloxycarbonyl, fluorenyloxycarbonyl and the like; preference is given to phenoxycarbonyl.

The term “heteroaryloxy” denotes heteroaryl radicals bonded via an oxygen atom and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are pyrrolyloxy, furanyloxy, thienyloxy, pyrazolyloxy, imidazolyloxy, oxazolyloxy, thiazolyloxy, pyridyloxy, pyrazinyloxy, pyridazinyloxy, pyrimidyloxy and the like; preferably pyrazolyloxy or pyridyloxy.

The term “heteroaryloxycarbonyl” denotes heteroaryloxy radicals bonded via a carbonyl group and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are pyrrolyloxycarbonyl, furanyloxycarbonyl, thienyloxycarbonyl, pyrazolyloxycarbonyl, imidazolyloxycarbonyl, oxazolyloxycarbonyl, thiazolyloxycarbonyl, pyridyloxycarbonyl, pyrazinyloxycarbonyl, pyridazinyloxycarbonyl, pyrimidyloxycarbonyl and the like; preferably imidazolyloxycarbonyl or oxazolyloxycarbonyl.

The term “arylalkyloxy” denotes arylalkyl radicals bonded via an oxygen atom, where the alkyl radical optionally bears 1, 2 or 3 substituents selected from halogen, cycloalkyl, alkoxy, haloalkoxy, and where the aryl group optionally bears 1, 2, 3 or 4 substituents selected from halogen, alkyl, haloalkyl, alkoxy and haloalkoxy. Examples thereof are benzyloxy, 2-phenylethyloxy (phenethyloxy) and the like; preference is given to benzyloxy.

The term “cycloalkylimino” denotes a radical of the formula —N═R bonded via the nitrogen, in which R represents cycloalkylidene radicals optionally bearing 1, 2 or 3 substituents selected from halogen, alkyl, haloalkyl, cycloalkyl, alkoxy, haloalkoxy. Examples of R are cyclopentylidene, cyclohexylidene, cycloheptylidene and the like.

The term “haloalkylimino” denotes a radical of the formula —N═R bonded via the nitrogen, in which R represents haloalkylidene radicals, where some or all of the hydrogen atoms in these straight-chain or branched alkylene groups are replaced by halogen atoms. Examples of R are chloromethylene, bromomethylene, dichloromethylene, fluoromethylene, difluoromethylene, chlorofluoromethylene, 1-chloroethylene, 1-bromoethylene, 1-fluoroethylene, 2-fluoroethylene, 2,2-difluoroethylene, 2,2,2-trifluoroethylene, 2-chloro-2-fluoroethylene, 2-chloro-2,2-difluoroethylene, 2,2-dichloro-2-fluoroethylene, 2,2,2-trichloroethylene and the like.

The term “cycloalkylcarbonyloxy” denotes cycloalkyl radicals bonded via a carbonyloxy group and having 3 to 10 carbon atoms as ring members, where the cycloalkyl radical optionally bears 1, 2 or 3 substituents selected from halogen, alkyl, haloalkyl, cycloalkyl, alkoxy and haloalkoxy. Examples thereof are cyclopropylcarbonyloxy, cyclobutylcarbonyloxy, cyclopentylcarbonyloxy, cyclohexylcarbonyloxy, cycloheptylcarbonyloxy, cyclooctylcarbonyloxy, cyclononylcarbonyloxy and the like, preferably cyclopentylcarbonyloxy or cyclohexylcarbonyloxy.

The term “arylalkylcarbonyloxy” denotes arylalkyl radicals bonded via a carbonyloxy group, where the alkyl radical optionally bears 1, 2 or 3 substituents selected from halogen, cycloalkyl, alkoxy, haloalkoxy, and where the aryl group optionally bears 1, 2, 3 or 4 substituents selected from halogen, alkyl, haloalkyl, alkoxy and haloalkoxy. Examples thereof are benzylcarbonyloxy, 2-phenylethylcarbonyloxy (phenethylcarbonyloxy) and the like, preferably benzylcarbonyloxy.

The term “arylcarbonyloxy” denotes aryl radicals bonded via a carbonyloxy group, where the aryl group optionally bears 1, 2, 3 or 4 substituents selected from halogen, alkyl, haloalkyl, alkoxy and haloalkoxy. Examples thereof are phenylcarbonyloxy, naphthylcarbonyloxy, fluorenylcarbonyloxy, anthracenylcarbonyloxy and the like; preferably phenylcarbonyloxy.

The term “heteroarylcarbonyloxy” denotes heteroaryl radicals bonded via a carbonyloxy group and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are 2-pyrrolylcarbonyloxy, 2-furanylcarbonyloxy, 2-thienylcarbonyloxy, 3-pyrazolylcarbonyloxy, 2-imidazolylcarbonyloxy, 2-oxazolylcarbonyloxy, 2-thiazolylcarbonyloxy, 4-triazolylcarbonyloxy, 4-pyridylcarbonyloxy and the like.

The term “heteroarylcarbonyl” denotes heteroaryl radicals bonded via a carbonyl group and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are 2-pyrrolylcarbonyl, 2-furanylcarbonyl, 2-thienylcarbonyl, 3-pyrazolylcarbonyl, 2-imidazolylcarbonyl, 2-oxazolylcarbonyl, 2-thiazolylcarbonyl, 4-triazolylcarbonyl, 4-pyridylcarbonyl and the like.

The term “alkylthio” denotes alkyl radicals bonded via a sulfur atom and having 1 to 10 carbon atoms (C₁-C₁₀-alkylthio), more preferably 1 to 6 carbon atoms (C₁-C₆-alkylthio), particularly 1 to 4 carbon atoms (C₁-C₄-alkylthio) and especially 1 to 3 carbon atoms (C₁-C₃-alkylthio), where the alkyl radical optionally bears 1, 2 or 3 substituents selected from halogen, alkyl, haloalkyl, cycloalkyl, alkoxy and haloalkoxy. Examples thereof are methylthio, ethylthio, n-propylthio, 1-methylethylthio (isopropylthio), n-butylthio, 1-methylpropylthio (sec-butylthio), 2-methylpropylthio (isobutylthio) and 1,1-dimethylethylthio (tert-butylthio) and the like; preferably methylthio, ethylthio and n-propylthio.

The term “haloalkylthio” describes haloalkyl groups bonded via a sulfur atom and having 1 to 10 carbon atoms (C₁-C₁₀-haloalkylthio), more preferably 1 to 6 carbon atoms (C₁-C₆-haloalkylthio), particularly 1 to 4 carbon atoms (C₁-C₄-haloalkylthio) and especially 1 to 3 carbon atoms (C₁-C₃-haloalkylthio). Examples thereof are chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, 1,1,2,2-tetrafluoroethylthio, 1-chloro-1,2,2-trifluoroethylthio, pentafluoroethylthio, 3,3,3-trifluoroprop-1-ylthio, 1,1,1-trifluoroprop-2-ylthio, 3,3,3-trichloroprop-1-ylthio, 1-chlorobutylthio, 2-chlorobutylthio, 3-chlorobutylthio, 4-chlorobutylthio, 1-fluorobutylthio, 2-fluorobutylthio, 3-fluorobutylthio, 4-fluorobutylthio and the like; preference is given to fluoromethylthio, 2-fluoroethylthio and trifluoromethylthio.

The term “cycloalkylthio” denotes cycloalkyl radicals bonded via a sulfur atom and having 3 to 10 carbon atoms as ring members (C₃-C₁₀-cycloalkylthio), preferably 3 to 6 carbon atoms (C₃-C₆-cycloalkylthio). Examples are cyclopropylthio, cyclobutylthio, cyclopentylthio, cyclohexylthio, cycloheptylthio, cyclooctylthio, cyclononylthio and cyclodecylthio. The cycloalkyl radicals may bear 1, 2 or 3 substituents selected from alkyl and halogen. Preferred cycloalkylthio radicals are cyclopentylthio or cyclohexylthio.

The term “arylthio” denotes aryl radicals bonded via a sulfur atom, where the aryl group optionally bears 1, 2, 3 or 4 substituents selected from halogen, alkyl, haloalkyl, alkoxy and haloalkoxy. Examples thereof are phenylthio, naphthylthio, fluorenylthio and the like; preference is given to phenylthio.

The term “heteroarylthio” denotes heteroaryl radicals bonded via a sulfur atom and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are 2-pyrrolylthio, 3-furanylthio, 3-thienylthio, 2-pyridylthio and the like; preferably 2-pyridylthio and 4-pyridylthio.

The term “arylalkylthio” denotes arylalkyl radicals bonded via a sulfur atom, where the alkyl radical optionally bears 1, 2 or 3 substituents selected from halogen, cycloalkyl, alkoxy and haloalkoxy, and where the aryl group optionally bears 1, 2, 3 or 4 substituents selected from halogen, alkyl, haloalkyl, alkoxy and haloalkoxy. Examples thereof are benzylthio, 2-phenylethylthio and the like; preference is given to benzylthio.

The term “cycloalkylsulfonyl” denotes cycloalkyl radicals bonded via a sulfonyl group (SO₂) and having 3 to 10 carbon atoms as ring members (C₃-C₁₀-cycloalkylsulfonyl), preferably 3 to 6 carbon atoms (C₃-C₆-cycloalkylsulfonyl), where the cycloalkyl radical optionally bears 1, 2 or 3 substituents selected from halogen, alkyl, haloalkyl, cycloalkyl, alkoxy and haloalkoxy. Examples thereof are cyclopropylsulfonyl, cyclobutylsulfonyl, cyclopentylsulfonyl, cyclohexylsulfonyl and the like; preferably cyclopropylsulfonyl, cyclopentylsulfonyl and cyclohexylsulfonyl.

The term “arylsulfonyl” denotes aryl radicals bonded via a sulfonyl group (SO₂), where the aryl group optionally bears 1, 2, 3 or 4 substituents selected from halogen, alkyl, haloalkyl, alkoxy and haloalkoxy. Examples thereof are phenylsulfonyl, naphthylsulfonyl, fluorenylsulfonyl and the like; preference is given to phenylsulfonyl.

The term “heteroarylsulfonyl” denotes heteroaryl radicals bonded via a sulfonyl group (SO₂) and having 1 to 4 heteroatoms selected from O, N, S and SO₂, where the heteroaryl group optionally bears 1, 2, 3 or 4 substituents selected from halogen, alkyl, haloalkyl, alkoxy and haloalkoxy. Examples thereof are 2-pyrrolylsulfonyl, 2-furanylsulfonyl, 2-thienylsulfonyl, 3-pyrazolylsulfonyl, 2-imidazolylsulfonyl, 2-oxazolylsulfonyl, 4-pyridylsulfonyl and the like; preferably 2-pyrrolylsulfonyl, 2-furanylsulfonyl and 4-pyridylsulfonyl.

The term “arylalkylsulfonyl” denotes arylalkyl radicals bonded via a sulfonyl group (SO₂), where the alkyl radical optionally bears 1, 2 or 3 substituents selected from halogen, cycloalkyl, alkoxy and haloalkoxy, and where the aryl group optionally bears 1, 2, 3 or 4 substituents selected from halogen, alkyl, haloalkyl, alkoxy and haloalkoxy.

Examples thereof are benzylsulfonyl, 2-phenylethylsulfonyl and the like; preference is given to benzylsulfonyl.

The details given hereinafter regarding preferred configurations of the processes according to the invention, especially regarding preferred configurations of the radicals in the various reactants and products and the reaction conditions of the processes according to the invention, apply either taken alone or, more particularly, in any conceivable combination with one another.

The reactions described herein are performed in reaction vessels customary for such reactions, and the reaction regime can be configured in a continuous, semicontinuous or batchwise manner. In general, the respective reactions will be performed under atmospheric pressure. The reactions can, however, also be performed under reduced (e.g. 0.1 to 1.0 bar) or elevated pressure (e.g. >1.0 to 100 bar).

More particularly, it is preferable to combine the embodiments with one another in any desired combination.

In the context of the present invention, m is preferably 0, 1, 2, 3 or 4, especially 0, 1, 2 or 3, more preferably 0, 1 or 2.

In the context of the present invention, n is preferably 1, 2, 3, 4 or 5, especially 1, 2 or 3.

In the context of the present invention, q is preferably 1, 2, 3 or 4, especially 1, 2 or 3.

In the context of the present invention, R¹ is preferably halogen, alkyl, hydroxyalkyl, haloalkyl, alkoxy, haloalkoxy, nitro, cyano, aryl, aryloxy or heteroaryl. More preferably, R¹ is halogen, alkyl, haloalkyl, alkoxy, haloalkoxy or optionally halogen-, alkyl- or alkoxy-substituted aryloxy, more preferably methyl, CF₃, chlorine, bromine, fluorine, alkoxy, haloalkoxy or phenoxy, and even more preferably methyl, CF₃, chlorine, bromine, fluorine, methoxy or OCF₃. More particularly, R¹ is 2-Me, 3-Me, 4-Me, 2-F, 3-F, 4-F, 2-Cl, 3-Cl, 4-Cl, 2-Br, 3-Br, 4-Br, 2-methoxy, 3-methoxy, 4-methoxy, 2-CF₃, 3-CF₃, 4-CF₄, 2-OCF₃, 3-OCF₃, or 4-OCF₃. R¹ is especially chlorine, bromine, fluorine or methoxy, and even more especially 2-F, 3-F, 4-F, 2-Cl, 3-Cl, 4-Cl, 2-Br, 3-Br, 4-Br, 2-methoxy, 3-methoxy or 4-methoxy. The stated positions are based on the 1 position via which the aryl radical which derives from the compound of the formula 1 is bonded to the aniline ring of the compound of the formula 3, or on the 1 position of the diazonium radical in the compound of the formula 1.

In the context of the present invention, X⁻ is preferably a halide, such as fluoride, chloride, bromide, iodide, BF₄⁻, PF₆⁻, hydrogensulfate, sulfate (½ SO₄²⁻), acetate, the anion of an aromatic 1,2-dicarboximide or the anion of an aromatic 1,2-disulfonimide. In the latter two cases, the anion forms through abstraction of the proton on the imide nitrogen atom. More preferably X⁻ is a halide, such as chloride or bromide, BF₄⁻ or sulfate (½ SO₄²⁻).

In the context of the present invention, Y⁻ is preferably a halide, such as fluoride, chloride, bromide, iodide, BF₄⁻, PF₆⁻, hydrogensulfate, sulfate (½ SO₄²⁻), acetate, the anion of an aromatic 1,2-dicarboximide or the anion of an aromatic 1,2-disulfonimide. In the latter two cases, the anion forms through abstraction of the proton on the imide nitrogen atom. More preferably Y⁻ is a halide, such as chloride or bromide, BF₄²⁻ or sulfate (½ SO₄²⁻).

In the context of the present invention, R² is preferably hydrogen, alkyl, haloalkyl, hydroxyalkyl, cycloalkyl, aryl, heteroaryl, arylalkyl or heteroarylalkyl. More preferably, R² is hydrogen or C₁-C₆ alkyl; especially hydrogen.

In the context of the present invention, R³ is preferably hydrogen, alkyl, haloalkyl, hydroxyalkyl, cycloalkyl, aryl, heteroaryl, arylalkyl or heteroarylalkyl. More preferably, R³ is hydrogen or C₁-C₆ alkyl; especially hydrogen.

Further preferably, R² and R³, together with the nitrogen atom to which they are bonded, form a 5- or 6-membered ring which may comprise 1 or 2 further heteroatoms as ring members, selected from O, S and N.

Further preferably, R² and R³ together form an alkylidene radical.

Most preferably, R² and R³ are each hydrogen atoms.

In the context of the present invention, R⁴ is preferably hydrogen, alkyl, haloalkyl, cycloalkyl, arylalkyl, aryl or heteroaryl.

In the context of the present invention, R⁵ is preferably hydrogen, alkyl, haloalkyl, cycloalkyl, arylalkyl, aryl or heteroaryl.

In the context of the present invention, R¹⁰ is preferably hydrogen, halogen, alkyl, haloalkyl, alkoxy, haloalkoxy, cycloalkyl, aryl or heteroaryl. More preferably, R¹⁰ is hydrogen, halogen, C₁-C₆ alkyl or C₁-C₆ haloalkyl. Especially preferably, R¹⁰ is hydrogen.

In the context of the present invention, R¹¹ is preferably hydrogen, halogen, hydroxy, cyano, aryl or heteroaryl.

In the context of the present invention, R¹⁴ is preferably hydrogen, alkyl or haloalkyl.

In the context of the present invention, R¹⁵ is preferably hydrogen, alkyl, haloalkyl, cyano, aryl or heteroaryl.

In the context of the present invention, R⁶ is preferably hydrogen, halogen, alkyl, haloalkyl, cycloalkyl, alkoxy, cyano, haloalkoxy, cycloalkoxy, alkylcarbonyloxy, haloalkylcarbonyloxy, aryloxy, aryl or heteroaryl. Particularly preferred for R⁶ are: hydrogen, fluorine, chlorine, bromine, cyano, methyl, ethyl, methoxy or ethoxy. More preferably, R⁶ is hydrogen, fluorine, chlorine, bromine, methoxy, CN or ethoxy. Alternatively, R⁶ is more preferably hydrogen, halogen, alkyl, haloalkyl, cycloalkyl, cyano, aryl or heteroaryl, and more preferably hydrogen, fluorine, chlorine, bromine, cyano, methyl or ethyl. More particularly, R⁶ is hydrogen, fluorine, chlorine, bromine or CN.

More preferably, R¹ is fluorine, chlorine, bromine or methoxy, R², R³ and R¹⁰ are each hydrogen, R⁶ is hydrogen, fluorine, chlorine, bromine, CN, methoxy or ethoxy, and preferably hydrogen, fluorine, chlorine, bromine or CN, and at the same time m is 0, 1, 2 or 3.

The process according to the invention is performed within the “basic range”; in other words, the reaction medium in which the reaction of 1 and 2 takes place is basic. Preferably, the reaction is performed at a pH of at least 9.1 (e.g. 9.1 to 14 or higher, e.g. to 14.5 or to 15), more preferably at least 9.5 (e.g. 9.5 to 14 or higher, e.g. to 14.5 or to 15), even more preferably at least 10 (e.g. 10 to 14 or higher, e.g. to 14.5 or to 15), yet more preferably at least 12 (e.g. 12 to 14 or higher, e.g. to 14.5 or to 15), particularly at least 13 (e.g. 13 to 14 or higher, e.g. to 14.5 or to 15), and especially at least 14 (e.g. 14 to 14.5 or 14 to 15). The pH may be greater than 14 when, for example, highly concentrated solutions of strong bases are used, for example a more than 1 molar solution of NaOH or KOH in water. The upper limit in this case is determined by the solubility of the base in the solvent (especially water).

The pH can be determined by means of customary methods, for example by means of indicators or standard pH meters, for example with glass electrodes or hydrogen electrodes, or with field-effect transistors. Typically, however, the pH is determined in a simple manner via the concentration of the base used, without taking activities into account.

Stated pH values are typically based on aqueous media, i.e. on the concentration/activity of an acid or base in water. If the reaction medium in which the reaction of 1 and 2 takes place is aqueous, the pH values are determined in the generally customary manner. If the reaction medium, in contrast, is nonaqueous, “within the basic range” in the context of the present invention means that the reaction medium in question comprises one or more bases in such a concentration that a purely aqueous medium (i.e. with water as the sole solvent) which comprised the same base(s) in the same concentration would be basic and would preferably have a pH of at least 9.1 (e.g. 9.1 to 14 or higher, e.g. to 14.5 or to 15), more preferably at least 9.5 (e.g. 9.5 to 14 or higher, e.g. to 14.5 or to 15), even more preferably at least 10 (e.g. 10 to 14), yet more preferably at least 12 (e.g. 12 to 14 or higher, e.g. to 14.5 or to 15), particularly at least 13 (e.g. 13 to 14 or higher, e.g. to 14.5 or to 15), and especially at least 14 (e.g. 14 to 14.5 or 14 to 15).

“Reaction medium” in this context means the medium in which the reaction of 1 and 2 takes place. This generally comprises, as well as 1 and 2, at least one solvent.

The aniline compound 2 is basic. However, the basicity thereof is generally insufficient, especially not when the pH is to be at least 9.1, and so the reaction of 1 and 2 is preferably performed in the presence of an (additional) base.

In the context of the processes according to the invention, suitable bases are, for example, inorganic bases such as alkali metal hydroxides, e.g. lithium, sodium or potassium hydroxide, alkaline earth metal hydroxides, e.g. magnesium or calcium hydroxide, aluminum hydroxide, alkali metal and alkaline earth metal hydroxides, for example sodium, magnesium or calcium oxide, alkali metal and alkaline earth metal carbonates, e.g. lithium, sodium, potassium or calcium carbonate, alkali metal and alkaline earth metal hydrogencarbonates, e.g. lithium, sodium or potassium hydrogencarbonate, or alkali metal and alkaline earth metal phosphates, e.g. lithium, sodium or potassium phosphate. Also suitable in principle are organic bases, such as alkoxides, e.g. sodium methoxide, sodium ethoxide, sodium tert-butoxide or potassium tert-butoxide and the like; and basic nitrogen heterocycles, such as pyridine or lutidine, preference being given to the alkoxides because of the higher basicity thereof. Preference is given to the inorganic bases mentioned, among which preference is given to the alkali metal hydroxides, alkaline earth metal hydroxides, alkali metal carbonates and alkali metal phosphates, and especially to the alkali metal and alkaline earth metal hydroxides mentioned, particularly alkali metal hydroxides such as lithium, sodium or potassium hydroxide; particularly sodium or potassium hydroxide; preferably in the form of the aqueous solution thereof.

In a preferred embodiment, the alkali metal and alkaline earth metal hydroxides are used in dilute form in aqueous solution. “Dilute” in this context means that the concentration of the base is 0.1 to 50% by weight, particularly 1 to 32% by weight and especially 2 to 16% by weight, based on the total weight of the solvent.

Aqueous bases are understood to mean a solution or dispersion of the bases mentioned in water.

An aqueous solution or aqueous medium in the context of the present invention is understood to mean a solution or a medium comprising a solvent or dispersant, the solvent or dispersant comprising water in a not insignificant amount in technical terms, for example in an amount of at least 10% by weight, preferably at least 20% by weight, more preferably at least 30% by weight, even more preferably at least 40% by weight and especially at least 50% by weight, based on the total weight of the solvent or dispersant. When the solvent or dispersant does not consist exclusively of water, it additionally comprises at least one solvent other than water. Suitable solvents are the water-miscible organic solvents listed below.

Accordingly, the aqueous solutions of the abovementioned inorganic and/or organic bases can also be used in a mixture with the water-miscible organic solvents specified below. In a particular embodiment, the concentration of the base in the aqueous solvent or solvent system is selected such that the pH of the reaction mixture is 9.1 or greater, preferably 9.5 or greater, more preferably 10 or greater, even more preferably 12 or greater, particularly 13 or greater and especially 14 or greater (e.g. 9.1 to 14 or to 14.5 or to 15; 9.5 to 14 or to 14.5 or to 15; 10 to 14 or to 14.5 or to 15; 12 to 14 or to 14.5 or to 15; 13 to 14 or to 14.5 or to 15; 14 to 14.5 or to 15).

It is likewise preferable to use mixtures of at least two of the bases mentioned when the resultant pH of the reaction mixture is 9.1 or greater, preferably 9.5 or greater, more preferably 10 or greater, even more preferably 12 or greater, particularly 13 or greater and especially 14 or greater (e.g. 9.1 to 14 or to 14.5 or to 15; 9.5 to 14 or to 14.5 or to 15; 10 to 14 or to 14.5 or to 15; 12 to 14 or to 14.5 or to 15; 13 to 14 or to 14.5 or to 15; 14 to 14.5 or to 15).

The reaction of the compounds of the formulae 1 and 2 can be performed either in a solvent or in substance. In the latter case, for example, the compound of the formula 2 itself functions as the solvent or dispersant or, if its melting point is above room temperature (25° C.), is initially charged as a melt and then admixed with the compound of the formula 1 under suitable reaction conditions. The preferred embodiment, however, is performance in a solvent preferably comprising at least one base.

Suitable solvents are aqueous solvents and organic solvents. Suitable organic solvents are, for example, short-chain nitriles, such as acetonitrile or propionitrile, amides such as N,N-dimethylformamide or N,N-dimethylacetamide, short-chain mono- or polyhydric alcohols such as methanol, ethanol, propanol, ethylene glycol or trifluoroethanol, dimethyl sulfoxide, open-chain and cyclic ethers such as diethyl ether, dioxane or tetrahydrofuran, sulfur compounds such as carbon disulfide or sulfolane, nitro compounds such as nitromethane, chloroalkanes such as dichloromethane or chloroform, open-chain and cyclic hydrocarbons such as pentane, hexane, heptane, benzine, petroleum ether or cyclohexane, or mixtures of these organic solvents with one another. Preferred organic solvents are short-chain nitriles such as acetonitrile or propionitrile, amides such as N,N-dimethylformamide or N,N-dimethylacetamide, short-chain mono- or polyhydric alcohols such as methanol, ethanol, propanol, ethylene glycol or trifluoroethanol, dimethyl sulfoxide and mixtures of these solvents. Particular preference is given to acetonitrile.

Suitable solvents among those mentioned above are especially those solvents or solvent systems which do not have any readily abstractable hydrogen atoms, since they give best possible protection to an aryl radical formed from side reactions.

Examples of solvents or solvent systems which do not have any readily abstractable hydrogen atoms are water, but also alcohols lacking hydrogen atoms in the a position, such as tert-butanol, particularly in a mixture with water, and some comparatively inert organic solvents or solvent systems, for example acetonitrile, trifluoroethanol and/or dimethyl sulfoxide. Especially an addition of water generally has a stabilizing effect on the aryl radicals formed, since these enter into virtually no side reactions with water. Water or aqueous solutions are therefore preferred as solvents without abstractable hydrogen atoms.

When solvents having readily abstractable hydrogen atoms, such as primary alcohols, are used, they are preferably used in a mixture with at least one further solvent which does not have any readily abstractable hydrogen atoms. Preferably, the organic solvents or solvent systems which are not inert toward the aryl radical are present in this case in an amount of not more than 50% by weight, more preferably of not more than 20% by weight and especially of not more than 10% by weight, based on the total weight of the solvent or solvent system. Since the solvents or solvent systems lacking readily abstractable hydrogen atoms used are especially water or aqueous solutions, the solvents or solvent systems used in the mixtures are preferably those which are miscible with water.

Overall, the solvents or solvent systems used are preferably water or mixtures of the abovementioned organic, water-miscible solvents or solvent systems with water or the aqueous bases mentioned.

As an alternative, not the pure solvents but mixtures which combine the solvent properties are used, or solubilizers are employed.

The term “solubilizer” denotes (interface-active) substances which, through their presence, make other compounds which are virtually insoluble in a solvent or solvent system soluble or emulsifiable in this solvent or solvent system, whether by entering into a molecular compound with the sparingly soluble substance or acting through micelle formation.

In a particularly preferred embodiment, aqueous solvents are used. Aqueous solvents are water or mixtures of water and at least one further solvent other than water. Solvents other than water are preferably organic solvents. Preferred organic solvents are water-miscible. Examples of water-miscible organic solvents are short-chain nitriles such as acetonitrile or propionitrile, amides such as N,N-dimethylformamide or N,N-dimethylacetamide, short-chain mono- or polyhydric alcohols such as methanol, ethanol, propanol, ethylene glycol or trifluoroethanol, and dimethyl sulfoxide. Particular preference is given to acetonitrile. Accordingly, particularly preferred solvents are water and aqueous solvents which, as well as water, comprise acetonitrile, i.e. water and water/acetonitrile mixtures.

Aqueous solvents which, as well as water, comprise at least one further solvent other than water comprise preferably 5 to 95% by weight, more preferably 10 to 95% by weight, even more preferably 20 to 95% by weight, yet more preferably and especially 30 to 95% by weight, and especially 40 to 95% by weight, of water, e.g. 50 to 90 or 60 to 90 or 70 to 90 or 75 to 85% by weight of water. The residual content corresponds to the further solvent(s).

In a preferred embodiment, the aqueous solvent or solvent system comprises a base, i.e. a base is present in the aqueous solvent or solvent system in a concentration of generally 0.1 to 50% by weight, particularly of 1 to 32% by weight and especially of 2 to 16% by weight, based on the total weight of the solvent.

Suitable and preferred bases are mentioned above. More particularly, sodium hydroxide or potassium hydroxide is used.

Also suitable in principle are nonaqueous solvents or solvent systems, for example the abovementioned organic solvents and mixtures of these solvents, but preference is given to the aqueous solvents.

In the case of use of nonaqueous solvents or solvent systems, a preferred embodiment in turn involves adding at least one of the bases mentioned to the nonaqueous solvent or solvent system.

“Solvent systems” are understood to mean a mixture of at least two solvents independently selected from the groups of aqueous organic and/or inorganic solvents. Preferably, water is one of the solvents used in the solvent system.

A further suitable solvent system is a biphasic solvent system comprising two essentially mutually immiscible solvents or solvent systems. “Essentially immiscible” means that a first solvent or solvent system which is used in a smaller amount than or the same amount as a second solvent or solvent system dissolves in the second solvent or solvent system to an extent of not more than 20% by weight, preferably to an extent of not more than 10% by weight and especially to an extent of not more than 5% by weight, based on the total weight of the first solvent or solvent system. Examples are systems which, as well as an above-defined aqueous solvent or solvent system, comprise one or more essentially water-immiscible solvents, such as carboxylic esters, e.g. ethyl acetate, propyl acetate or ethyl propionate, open-chain ethers such as diethyl ether, dipropyl ether, dibutyl ether, methyl isobutyl ether and methyl tert-butyl ether, aliphatic hydrocarbons such as pentane, hexane, heptane and octane, and also petroleum ether, halogenated aliphatic hydrocarbons such as methylene chloride, trichloromethane, dichloroethane and trichloroethane, cycloaliphatic hydrocarbons such as cyclopentane and cyclohexane, and aromatic hydrocarbons such as benzene, toluene, xylenes, chlorobenzene, dichlorobenzenes and mesitylene.

Preferably, one phase comprises at least one protic solvent such as water, the abovementioned alcohols or diols. More preferably, the first phase is an aqueous solvent or solvent system to which at least one base, such as sodium hydroxide, potassium hydroxide and the like, has been added, or a mixture of water and at least one base with at least one water-miscible organic solvent, for example alcohols such as methanol, ethanol, propanol or trifluoroethanol, diols such as ethylene glycol, acetonitrile, and amides such as N,N-dimethylformamide and N,N-dimethylacetamide. More particularly, the first phase comprises water or an aqueous solution of at least one of the bases mentioned, the base preferably being sodium hydroxide or potassium hydroxide.

The other phase is preferably selected from aliphatic hydrocarbons such as pentane, hexane, heptane and octane, and also petroleum ether, halogenated aliphatic hydrocarbons such as methylene chloride and 1,2-dichloroethane, and cycloaliphatic hydrocarbons such as cyclopentane and cyclohexane.

Such a biphasic solvent system may also comprise at least one phase transfer catalyst. Phase transfer catalysts are sufficiently well known to those skilled in the art and comprise, for example, charged systems such as organic ammonium salts, for example tetra(C₁-C₁₈-alkyl)ammonium chlorides or bromides, such as tetramethylammonium chloride or bromide, tetrabutylammonium chloride or bromide, hexadecyltrimethylammonium chloride or bromide, octadecyltrimethylammonium chloride or bromide, methyltrihexylammonium chloride or bromide, methyltrioctylammonium chloride or bromide or benzyltrimethylammonium hydroxide (triton B), and also tetra(C₁-C₁₈-alkyl)phosphonium chlorides or bromides, such as tetraphenylphosphonium chloride or bromide, [(phenyl)ₐ-(C₁-C₁₈-alkyl)_{b}]phosphonium chlorides or bromides in which a=1 to 3 and b=3 to 1 and the sum of a+b=4, and also pyridinium salts such as methylpyridinium chloride or bromide, and uncharged systems such as crown ethers or azo crown ethers, e.g. 12-crown-4,15-crown-5, 18-crown-6, dibenzo-18-crown-6 or [2.2.2]-cryptand (222-Kryptofix), cyclodextrins, calixarenes such as [14]-metacyclophane, calix[4]arene and p-tert-butylcalix[4]arene, and cyclophanes.

In the context of the process according to the invention, it is not necessary that the compound of the formula 2 is fully soluble in the solvent or solvent system used.

In a particular embodiment, the solvents or solvent systems are used in degassed form (i.e. specifically in oxygen-free form). The degassing of solvents or solvent systems is known and can be effected, for example, by single or multiple freezing of the solvent or solvent mixture, thawing under reduced pressure (for removal of the gas dissolved/dispersed in the solvent or solvent system) and compensation with an inert gas, such as nitrogen or argon. Alternatively or additionally, the solvent or solvent system can be treated with ultrasound. The latter procedure is an option especially for water or aqueous solvents or solvent systems, since the expansion of water on freezing can lead to apparatus problems.

The reaction of the compound of the formula 1 with the compound of the formula 2 is effected generally at a temperature in the range from −100° C. up to the boiling point of the reaction mixture, for example from −78° C. to 200° C. or from 0° C. to 150° C. Preference is given, however, to reaction at elevated temperature, preferably of 50° C. to 130° C. and especially of 60 to 110° C. These temperatures apply to performance in solution; if the experiment, in contrast, is conducted in substance and the melting point of the compound of the formula 2 is above room temperature, the reaction temperature of course corresponds at least to the temperature of the melt of the reaction mixture.

The process according to the invention is preferably performed in such a way that the compound of the formula 1 or the compound of the formula 2 or both compounds 1 and 2 are used in the reaction dispersed in an alkaline medium. If the compound of the formula 1 is dispersed in an alkaline medium, the compounds 1a/1b/1c are formed at first through the reaction of 1 with the base of the alkaline medium, and these are then reacted with the compound 2. If compound 2 is dispersed in an alkaline medium, the conversion of the compound 1 to the compounds 1a/1b/1c is effected on addition of 1 to the dispersion of the compound 2 in an alkaline medium.

The pH of the alkaline medium in this case is preferably at least 9.1 (e.g. 9.1 to 14 or higher, e.g. to 14.5 or to 15), more preferably at least 9.5 (e.g. 9.5 to 14 or higher, e.g. to 14.5 or to 15), even more preferably at least 10 (e.g. 10 to 14 or higher, e.g. to 14.5 or to 15), yet more preferably at least 12 (e.g. 12 to 14 or higher, e.g. to 14.5 or to 15), particularly at least 13 (e.g. 13 to 14 or higher, e.g. to 14.5 or to 15), and especially at least 14 (e.g. 14 to 14.5 or 14 to 15).

The reactants can in principle be contacted with one another in different sequences. For example, the compound of the formula 2, optionally dissolved or dispersed in a solvent or solvent system, or optionally dissolved or dispersed in an alkaline medium, can be initially charged and admixed with the compound of the formula 1, optionally dissolved or dispersed in a solvent or solvent system, or optionally dissolved or dispersed in an alkaline medium.

Conversely, the compound of the formula 1, which in this case must be dissolved or dispersed in an alkaline medium, can be initially charged and admixed with the compound of the formula 2, optionally dissolved or dispersed in a solvent or solvent system or optionally dissolved or dispersed in an alkaline medium. It is preferable in this case that the components are mixed under such conditions that the intermediates 1a/1b/1c formed under alkaline conditions from compound 1 essentially do not decompose before they can react with 2. More particularly, the components are mixed at sufficiently low temperatures at which essentially no decomposition of 1a/1b/1c takes place yet. The specifically suitable maximum temperatures depend here on the compound 1 used in each case. According to the compound 1 used, the components are mixed at a temperature of preferably not more than 50° C., e.g. −20 to 50° C. or 0 to 50° C., or at a temperature of preferably not more than 30° C., e.g. −20 to 30° C. or 0 to 30° C., or at a temperature of preferably not more than 25° C., e.g. −20 to 25° C. or 0 to 25° C., or at a temperature of preferably not more than 20° C., e.g. −20 to 20° C. or 0 to 20° C. The temperature can then, if desired, be increased after mixing.

In the case of performance in a biphasic system, it is alternatively possible to initially charge the compound of the formula 2 in the solvent or solvent system of one phase, and the compound of the formula 1 in the solvent or solvent system of the second phase.

It has been found to be advantageous, however, to initially charge the compound of the formula 2, optionally in a solvent or solvent system or optionally in an alkaline medium, and to add the compound of the formula 1, optionally dissolved or dispersed in a solvent or solvent system or optionally in an alkaline medium. At least one of the compounds 1 and 2 should be initially charged in an alkaline medium. In this case, the compound of the formula 1 is preferably added gradually (in portions or continuously). In many cases, the gradual addition suppresses the formation of homo-coupling products, i.e. of products which arise through reaction of two or more compounds of the formula 1 with one another, since a low concentration of the compound of the formula 1 in the reaction mixture ensures that the reaction thereof with the compound of the formula 2 predominates over the reaction with itself.

The rate of addition is determined by several factors, such as batch size, temperature, reactivity of the reactants and type of reaction conditions selected, this rate of addition bringing about decomposition of the compound of the formula 1a, 1b and/or 1c to nitrogen and an aryl radical, and can be determined by the person skilled in the art in the individual case, for example by suitable preliminary tests. For instance, a low reactivity of the reactants requires a relatively slow addition rate, but this can be at least partly compensated for, for example, by a higher temperature and/or by the selection of reaction conditions which accelerate decomposition of the compound of the formula 1a, 1b and/or 1c.

Compounds 1 and 2 are used in a molar ratio of preferably 1:1000 to 5:1, for example of 1:500 to 1:1. Particular preference is given, however, to using compound 1 in deficiency in relation to compound 2. More particularly, compounds 1 and 2 are used in a molar ratio of 1:2 to 1:50, more preferably of 1:3 to 1:20 and even more preferably of 1:5 to 1:20.

The two preferred measures, i.e. the use of the compound of the formula 1 in deficiency (based on the compound of the formula 2) and the stepwise addition thereof, bring about advantageous running of the reaction, since they suppress the homo-coupling of the compound of the formula 1.

Irrespective of the method of performance, the base is preferably used in at least an equimolar amount to the compound 1. Preferably, the molar ratio of base to compound 1 is 1:1 to 50:1, more preferably 2:1 to 20:1 and especially 3:1 to 10:1.

Preference is given to using the compound of the formula 2 directly as the free amine. Alternatively, it can also be used, either in full or in part, in the form of one of the acid adducts thereof or of a mixture of such adducts, particular preference being given to the hydrochloride of the compound of the formula 2. In the case of use of the acid adducts of the compound of the formula 2, it has to be ensured by addition of at least one base that the reaction (i.e. first the formation and then the decomposition of the compound of the formula 1a, 1b and/or 1c to nitrogen and an aryl radical) again proceeds within the basic range.

In a preferred embodiment, the compound of the formula 2 is initially charged in an alkaline medium and the compound of the formula 1 is added. In this case, preferably, the compound of the formula 2 is initially charged in the form of an aqueous dispersion comprising a base, and the compound of the formula 1 is added to this dispersion. Compound 1 in this case can be used in substance or in the form of a dispersion, especially in the form of the solution as formed in the preparation of the compound 1. The dispersion of the compound 1 here may also be acidic, in which case, however, the basicity of the initial charge must be sufficiently high that, in spite of the addition of the acidic dispersion of the compound 1, the required pH is complied with in the course of the reaction, i.e. the pH after addition of the acidic dispersion does not go below the desired value.

The term “dispersion” in the context of the present invention comprises any form of the mixture of a substance which can assume any state of matter and is generally liquid or solid with a solvent (also referred to as dispersant). Examples are especially suspensions, emulsions and solutions. Analogously, the term “dispersed” comprises a substance distributed in a solvent, for example suspended, emulsified or dissolved.

The pH of the initial charge (i.e. of the alkaline medium or of the aqueous dispersion comprising the compound 2) is preferably at least 9.1 (e.g. 9.1 to 14 or higher, e.g. to 14.5 or to 15), more preferably at least 9.5 (e.g. 9.5 to 14 or higher, e.g. to 14.5 or to 15), more preferably at least 10 (e.g. 10 to 14 or higher, e.g. to 14.5 or to 15), yet more preferably at least 12 (e.g. 12 to 14 or higher, e.g. to 14.5 or to 15), particularly at least 13 (e.g. 13 to 14 or higher, e.g. to 14.5 or to 15), and especially at least 14 (e.g. 14 to 14.5 or 14 to 15).

Suitable and preferred bases are mentioned above; more particularly, sodium hydroxide or potassium hydroxide are used.

Before addition of the compound of the formula 1, the initial charge is preferably heated, preferably to a temperature of 50 to 130° C., especially 60° C. to 110° C.

In an alternatively preferred embodiment, in a first step, the compound of the formula 1 is first reacted in aqueous medium with a base, and, in a second step, the dispersion obtained is added to the compound of the formula 2. In the first step, compound 1 reacts at least partly to give compounds 1a, 1b and/or 1c. It is assumed that these intermediates are also passed through in situ in the first performance variant (addition of compound 1 to the compound 2 initially charged in alkaline medium).

Compound 2 can be used in substance or in the form of a dispersion, for example of a solution in an organic solvent. When the compound 2 is liquid, it is preferable to use it in substance, i.e. without solvent. If it is used in the form of a dispersion/solution, suitable solvents are, for example, the abovementioned organic solvents and especially the abovementioned water-miscible organic solvents.

The pH of the aqueous medium in which the compound 1 is first converted is preferably at least 9.1 (e.g. 9.1 to 14 or higher, e.g. to 14.5 or to 15), more preferably at least 9.5 (e.g. 9.5 to 14 or higher, e.g. to 14.5 or to 15), more preferably at least 10 (e.g. 10 to 14 or higher, e.g. to 14.5 or to 15), yet more preferably at least 12 (e.g. 12 to 14 or higher, e.g. to 14.5 or to 15), particularly at least 13 (e.g. 13 to 14 or higher, e.g. to 14.5 or to 15), and especially at least 14 (e.g. 14 to 14.5 or 14 to 15).

Suitable and preferred bases are mentioned above; more particularly, sodium hydroxide or potassium hydroxide are used.

Before addition of the dispersion, the compound 2 is preferably heated, preferably to a temperature of 50 to 130° C., especially of 60 to 110° C.

The process according to the invention can also be performed under the following additional conditions/measures:
performance in the presence of at least one reducing agent;
performance under irradiation with electromagnetic radiation in the visible and/or ultraviolet range;
performance with employment of ultrasound;
performance under the conditions of an electrochemical reduction;
performance under radiolysis conditions;
performance using a combination of at least two of these measures.

The term “reducing agent” refers to those elements and compounds which, as electron donors [also electron-donor complexes], attempt to release electrons with conversion to a lower-energy state, particularly to form stable electron shells. A measure of the strength of a reducing agent is the redox potential. Examples of reducing agents are inorganic salts, metals, metal salts or reducing organic compounds.

In a formal sense, the hydroxide ions or alkoxide ions used for the establishment of the basic pH also act as reducing agents. However, the performance of the reaction in the presence of at least one reducing agent is understood in the context of the present invention to mean performance in the presence of a reducing agent other than the reducing agents inherently present, such as hydroxide ions or alkoxide ions.

When the reaction is performed in the presence of a reducing agent, it is preferably performed in such a way that the compound of the formula 2 and the reducing agent are initially charged, preferably dissolved or dispersed in a solvent or solvent system, and admixed gradually with the compound of the formula 1. With regard to addition rate, reaction temperature and solvent or solvent system, reference is made to the details which follow.

The at least one reducing agent is preferably selected from reducing metal salts, metals and/or reducing anions; however, suitable reducing agents also include others whose reduction potential is sufficiently great to transfer an electron to the compound of the formula 1 used in each case. This includes such different compounds as pyrene, ascorbic acid and hemoglobin. Preference is given, however, to the use of reducing metals, metal salts and/or reducing anions.

In the context of the present invention, it is possible to use any desired reducing metal salts, provided that the reduction potential thereof is sufficiently great to transfer an electron to the compound of the formula 1 used in each case. Reducing metal salts are understood in the context of the present invention to mean those in which the most stable oxidation number of the metal under the reaction conditions is higher than in the form used, such that the metal salt acts as a reducing agent.

Preferred metal salts are at least partly soluble in the reaction medium. Since the reaction medium is preferably aqueous, preferred reducing metal salts are correspondingly water-soluble. Preferred counterions of the metal salts are customary water-soluble anions, such as the halides, especially chloride, sulfate, nitrate, acetate and the like.

However, metal complexes are also suitable, such as hexacyanoferrate(II) or ferrocene.

Reducing metal salts are selected from Cu(I) salts, Fe(II) salts, tin(II) salts and vanadium(II) salts, and especially from Cu(I) salts and Fe(II) salts. Preferred among these are the water-soluble salts thereof, such as the chlorides, sulfates, nitrates, acetates and the like.

Preferred reducing metals are selected from iron, copper, cobalt, nickel, zinc, magnesium, titanium and chromium, more preferably iron and copper.

Preference is given to using the reducing metal(s) or metal salt(s) in a total amount of 0.005 to 8 mol, more preferably of 0.01 to 3 mol, even more preferably of 0.1 to 1 mol, based on 1 mol of the compound of the formula 1.

If the reaction is performed in degassed solvents or solvent systems (i.e. those freed of oxygen) and under an inert gas atmosphere, such as nitrogen or argon, the reducing metal salt can be used in smaller amounts, for example in an amount of 0.005 to 4 mol based on 1 mol of the compound of the formula 1.

Suitable reducing anions are, for example, bromide, iodide, sulfite, hydrogensulfite, pyrosulfite, dithionite, thiosulfate, nitrite, phosphite, hypophosphite, ArS⁻, xanthogenates (R′OCS2⁻; R′=alkyl, aryl), alkoxides such as methoxide, ethoxide, propoxide, isopropoxide, butoxide, isobutoxide and tert-butoxide, and phenoxide. The reducing anions are of course preferably selected from those whose reduction potential, also within the pH range selected, is still sufficient to bring about the decomposition of the compound of the formula 1a, 1b and/or 1c to an aryl radical and nitrogen.

The reducing anions are used in an amount of preferably 0.005 to 8 mol, more preferably of 0.01 to 6 mol and especially of 1 to 6 mol, based on 1 mol of the compound of the formula 1.

Alternatively or additionally, in a preferred embodiment of the process according to the invention, the procedure is effected under the conditions of an electrochemical reduction. In this procedure, aryldiazenyl radicals are generated by cathodic reduction from the compound of the formula 1, which initiates the decomposition of the abovementioned compounds.

The procedure is effected, for example, in such a way that cathode and anode are placed into the reaction vessel comprising the compound of the formula 2 initially charged in a suitable solvent or solvent system, and voltage is applied during the gradual addition of the compound of the formula 1. The voltage and current density to be selected depends on various factors, such as addition rate and solvent or solvent system, and has to be determined in the individual case, which is possible, for example, with the aid of preliminary tests. The solvents or solvent systems are suitably selected such that they enter into a minimum level of competing reactions at the electrodes under the given reaction conditions. Since the cathodic reduction of protons can be avoided only with difficulty even at very low current densities and voltage, preference is given to using aprotic polar solvents such as acetonitrile, dimethylformamide or acetone.

Alternatively or additionally, the process for preparing compounds of the structure 3 is effected by effecting the reaction under irradiation with electromagnetic radiation in the visible and/or ultraviolet region. Preference is given to using electromagnetic radiation having a wavelength in the range from 100 to 400 nm, more preferably in the range from 200 to 380 nm and especially in the range from 250 to 360 nm.

The procedure under irradiation is preferably effected in such a way that the compound of the formula 2 is initially charged in a suitable solvent or solvent system and is irradiated with cooling during the gradual addition of the compound of the formula 1. Especially when UV radiation is used, the solvents or solvent systems are preferably used in degassed form, since reactive oxygen species can otherwise form, and these can lead to unwanted products. Since the degassing of water or aqueous solutions is not trivial, the organic solvents mentioned below are an option in this case.

Alternatively or additionally, the process for preparing compounds of the formula 3 is effected by performing the reaction with application of ultrasound. Like all soundwaves, ultrasound too generates periodic compression and expansion of the medium; the molecules are compressed and expanded. Small bubbles form, which grow and immediately implode again. This phenomenon is called cavitation. Each imploding bubble sends out shockwaves and tiny liquid jets with a speed of about 400 km/h, which act on the immediate environment. Cavitation can be exploited, for example, in order to accelerate chemical reactions and increase the solubility of products in a particular medium.

The procedure with application of ultrasound can be effected, for example, in such a way that the reaction vessel in which the compound of the formula 2 has been initially charged in a suitable solvent or solvent system is within an ultrasound bath, and the reaction mixture is exposed to ultrasound during the gradual addition of the compound of the formula 1. Instead of the use of an ultrasound bath, it is possible to mount a sonotrode (=device which transmits the ultrasound vibrations produced by a sound transducer to the material to be treated with ultrasound) in the reaction vessel in which the compound of the formula 2 has been initially charged in a suitable solvent or solvent system. The latter alternative is an option especially for relatively large batches. With regard to addition rate, reaction temperature and solvent or solvent system, preliminary tests have to be conducted.

Alternatively or additionally, in a preferred embodiment of the process according to the invention, the procedure is effected under radiolysis conditions. In this case, solvated electrons are produced in aqueous solution by irradiation with γ radiation, for example from a ⁶⁰Co source. This procedure is described in detail in J. E. Packer et al., J. Chem. Soc., Perkin Trans. 2, 1975, 751 and in Aust. J. Chem. 1980, 33, 965, which are hereby fully incorporated by reference.

Of the aforementioned measures, the procedure in the presence of at least one reducing agent and especially of at least one reducing anion is preferred. Compounds of the formula 1 are common knowledge and can be prepared by standard processes, as described, for example, in Organikum, Wiley VCH, 22nd edition. For instance, they are obtainable by diazotization of the corresponding aniline derivative, for example by reacting such an aniline derivative with nitrite in the presence of an acid, for instance semiconcentrated sulfuric acid. Corresponding aniline derivatives for preparation both of compounds of the formula 1 and of compounds of the formula 2 are known or can be prepared by known processes, for example by hydrogenating or homogeneously reducing correspondingly substituted nitrobenzenes in the presence of a suitable catalyst (for instance Sn(II) chloride/HCl; cf. Houben Weyl, “Methoden d. org. Chemie” [Methods of Organic Chemistry] 11/1, 422). Preparation from azobenzenes and substitution of suitable benzenes with ammonia are also standard methods. The preparation of compounds of the formula 1 in which the counteranions are selected from the anions of aromatic dicarboximides or disulfonimides can be effected analogously to M. Barbero et al., Synthesis 1998, 1171-1175.

The workup of the reaction mixtures obtained and the isolation of the compounds of the formula 3 is effected in a customary manner, for example by an extractive workup, by removal of the solvent, for example under reduced pressure, or by a combination of these measures. A further purification can be effected, for example, by crystallization, distillation or by chromatography.

Excess or unconverted reactions (these are particularly the compound of the formula 2, which is preferably used in excess in relation to the compound of the formula 1) are preferably isolated in the course of workup and reused.

In accordance with a preferred embodiment of the invention, the reaction mixture is worked up by diluting it with water and extracting it repeatedly with a suitable, essentially water-immiscible organic solvent, and concentrating the combined organic phases. According to the acid-base properties of the product, the pH before the extraction is optionally set suitably by addition of acids or bases. Examples of suitable, essentially water-immiscible organic solvents have been listed above. The product thus isolated can subsequently be kept ready for uses or sent directly to a use, for example used in a further reaction step, or purified further beforehand.

The conversion of the compound 3 to the compound 10 is effected by customary prior art processes for amide formation.

For instance, the process for preparing the compound 10, in a preferred embodiment, also comprises the following step:

N-acylation of the compound 3 by reaction with a compound of the general formula 11,

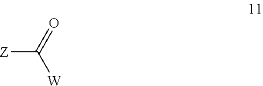

in which Z is as defined above, and W is a leaving group, to obtain a compound 10.

In the compounds of the formulae 3 and 11, Z is preferably 5- or 6-membered hetaryl having 1, 2 or 3 nitrogen atoms as ring members, where the hetaryl radical optionally bears 1, 2 or 3 substituents preferably selected from halogen, C₁-C₄-alkyl and C₁-C₄-haloalkyl. Preferably, the 5- or 6-membered hetaryl radical Y bears 1 or 2 substituents preferably selected from halogen, C₁-C₄-alkyl and C₁-C₄-haloalkyl.

The 5- or 6-membered hetaryl radical having 1, 2 or 3 nitrogen atoms as ring members is, for example, pyrrolyl such as 1-, 2- or 3-pyrrolyl, pyrazolyl such as 1-, 3-, 4- or 5-(1H)-pyrazolyl, imidazolyl such as 1-, 3-, 4- or 5-(1H)-imidazolyl, triazolyl such as 1-, 4- or 5-[1,2,3]-(1H)-triazolyl, 2- or 4-[1,2,3]-(2H)-triazolyl, pyridyl such as 2-, 3- or 4-pyridyl, pyrazinyl such as 2-pyrazinyl, pyrimidinyl such as 2-, 4- or 5-pyrimidinyl, pyridazinyl such as 3- or 4-pyridazinyl, or triazinyl such as 2-[1,3,5]-triazinyl. Preferably, the 5- or 6-membered hetaryl radical having 1, 2, or 3 nitrogen atoms as ring members is pyrazolyl such as 1-, 3-, 4- or 5-(1H)-pyrazolyl, or pyridyl such as 2-, 3- or 4-pyridyl, and especially pyrazol-4-yl or pyridin-3-yl.

Z is especially 2-chloropyrid-3-yl, 1-methyl-3-(trifluoromethyl)pyrazol-4-yl, 1-methyl-3-(difluoromethyl)pyrazol-4-yl or 1,3-dimethyl-5-fluorpyrazol-4-yl.

For the inventive N-acetylation of an aminobiphenyl of the formula 3, the reagent of the formula 11 used is generally a carboxylic acid or derivative of a carboxylic acid capable of amide formation, for instance an acid halide, acid anhydride or ester. Accordingly, the leaving group W is typically hydroxyl, halide, especially chloride or bromide, an —OR^{a} radical or an —O—CO—R^{b} radical.

If the compound 11 is used in the form of the carboxylic acid (Z—COOH; W═OH), the reaction can be performed in the presence of a coupling reagent. Suitable coupling reagents (activators) are known to those skilled in the art and are selected, for example, from carbodiimides such as DCC (dicyclohexylcarbodiimide) and DCI (diisopropylcarbodiimide), benzotriazole derivates such as HBTU ((O-benzotriazol-1-yl)-N,N′,N′-tetramethyluroniumhexafluorophosphate) and HCTU (1-[bis(dimethylamino)methylene]-5-chloro-1H-benzotriazolium tetrafluoroborate), and phosphonium activators such as BOP ((benzotriazol-1-yloxy)tris(dimethylamino)phosphonium hexafluorophosphate), Py-BOP ((benzotriazol-1-yloxy)tripyrrolidinephosphonium hexafluorophosphate) and Py-BrOP (bromotripyrrolidinephosphonium hexafluorophosphate). In general, the activator is used in excess. The benzotriazole and phosphonium coupling reagents are generally used in a basic medium.

Suitable derivatives of the carboxylic acid Z—COOH are all derivatives which can react with the aminobiphenyl 3 to give the amide 10, for example esters Z—C(O)—OR^{a} (W═OR^{a}), acid halides Z—C(O)X, in which X is a halogen atom (W=halogen), or acid anhydrides Y—CO—O—OC—R^{b} (W=—O—CO—R^{b}).

The acid anhydride Z—CO—O—OC—R^{b} is either a symmetric anhydride Z—CO—O—OC—Z (R^{b}═Z), or an asymmetric anhydride in which —O—OC—R^{b} is a group which can be displaced readily by the aminobiphenyl 3 used in the reaction. Suitable acid derivatives with which the carboxylic acid Z—COOH can form suitable mixed anhydrides are, for example, the esters of chloroformic acid, e.g. isopropyl chloroformate and isobutyl chloroformate, or of chloroacetic acid.

Suitable esters Z—COOR^{a} preferably derive from C₁-C₄-alkanols R^{a}OH in which R^{a} is C₁-C₄-alkyl, such as methanol, ethanol, propanol, isopropanol, n-butanol, butan-2-ol, isobutanol and tert-butanol, preference being given to the methyl and ethyl esters (R^{a}=methyl or ethyl). Suitable esters can also derive from C₂-C₆-polyols such as glycol, glycerol, trimethylolpropane, erythritol, pentaerythritol and sorbitol, preference being given to the glyceryl ester. When polyol esters are used, it is possible to use mixed esters, i.e. esters having different R^{a} radicals.

Alternatively, the ester Z—COOR^{a} is what is called an active ester, which is obtained in a formal sense by the reaction of the acid Z—COOH with an active ester-forming alcohol such as p-nitrophenol, N-hydroxybenzotriazole (HOBt), N-hydroxysuccinimide or OPfp (pentafluorophenol).

Alternatively, the reagent 11 used for N-acylation may have another commonly used leaving group W, for example thiophenyl or imidazolyl.

The inventive N-acylations with the above-described reagents of the formula 11 can be performed analogously to known processes.

For the N-acylation of compounds 3, preference is given to using carbonyl halides 11, especially those in which the leaving group W is chlorine or bromine, and is more preferably chlorine. For this purpose, preferably 0.5 to 4 mol and especially 1 to 2 mol of the acid chloride are used per 1 mol of the compound 3.

Typically, the N-acylation of an aminobiphenyl 3 is performed with an acid chloride 11 in the presence of a base, for instance triethylamine, using generally 0.5 to 10 mol, especially 1 to 4 mol, of the base per 1 mol of the acid chloride.

Frequently, a compound of the formula 10 will be prepared by initially charging the corresponding compound 3 together with the base, preferably in a solvent, and adding the acid chloride, optionally dissolved in a solvent, stepwise at a temperature in the range from about −30° C. to 50° C., especially from 0° C. to 25° C. Typically, reaction is subsequently allowed to continue at elevated temperature, for instance in the range from 0° C. to 150° C., especially from 15° C. to 80° C.

The acylation can, however, also be performed in the absence of a base. For this purpose, the acylation is performed in a biphasic system. In this case, one of the phases is aqueous and the second phase is based on at least one essentially water-immiscible organic solvent. Suitable aqueous solvents and suitable essentially water-immiscible organic solvents have been described above and also in WO 03/37868. This reference, in which further suitable reaction conditions for acylation processes in the absence of bases are also described in general terms, is hereby fully incorporated by reference.

If R¹ or R⁶ in compounds 3 is an amino group, or R⁶ or R¹⁰ comprise an amino group, it is necessary for selective preparation of compounds 10 to protect this amino group before the reaction, in order to prevent the acylation from proceeding on the nitrogen atom of this group. Suitable protecting groups and processes for introduction thereof are known to those skilled in the art. For example, the compound 3 can be converted by reaction with Boc anhydride to a compound 3 in which the amino group to be protected has been protected with tert-butoxycarbonyl. The compound 3 can be converted by reaction with acetyl chloride to a compound 3 in which the amino group to be protected has been protected with acetyl. The compound 3 can be converted by reaction with dimethylformamide in the presence of POCl₃ or thionyl chloride to a compound 3 in which the amino group to be protected has been protected as N═C—N(CH₃)₂. The compound 3 can be converted by reaction with allyl chloride to a compound 3 in which the amino group to be protected has been protected as N(CH₂—CH═CH₂)₂. The compound 3 can be converted by reaction with an aliphatic or aromatic aldehyde to a compound 3 in which the amino group to be protected has been protected as N═C—R in which R is C₁-C₃-alkyl or aryl such as phenyl. The compound 3 can be converted by reaction with a C₁-C₄-alkyl- or arylsulfonyl chloride, especially with methylsulfonyl chloride, to a compound 3 in which the amino group to be protected has been protected with C₁-C₄-alkylsulfonyl or arylsulfonyl and especially with methylsulfonyl. Since the introduction of the protecting group at the stage of compound 3 is not selective under some circumstances, it is more favorable in these cases to introduce the protecting group as early as before the biphenyl formation, and hence to use a compound 1 or 2 in which R¹ and/or R⁶ is a protected amino group or R⁶ and/or R¹⁰ comprise a protected amino group. In that case, the protecting group can be detached again if desired by means of known processes on completion of the acylation step, for example by hydrolysis, or, in the case of allyl protecting groups, by reaction with a base in the presence of palladium and a nucleophile such as malonic acid.

### EXAMPLES

Solvents and reagents were degassed with nitrogen before use. ¹H NMR spectra were recorded on 360 and 600 MHz spectrometers using CDCl₃ as a solvent with CHCl₃ (7.26 ppm) as a standard. Chemical shifts are reported as parts per million (ppm). Coupling constants are reported in Hertz (JHz). The following abbreviations are used for the description of the signals: s (singlet), d (doublet), dd (doublet of doublets), ddd (doublet of doublet of doublets), t (triplet), q (quadruplet), m (multiplet). ¹³C NMR spectra were recorded at 90.6 and 150.9 MHz in CDCl₃ with CHCl₃ (77.0 ppm) as a standard. Chemical shifts are reported as parts per million (ppm). ¹⁹F NMR spectra were recorded at 338.8 MHz in CDCl₃ with C₆F₆ (−164.9 ppm) as a standard. Mass spectra were recorded on a Jeol GC mate II GC-MS system with electron impact (EI). Analytical thin-layer chromatography (TLC) was conducted on Merck silica gel plates using short-wave (254 nm) UV. For flash chromatography, silica gel (silica gel 60, 40-63 μm, Merck) was used.

### Abbreviations:

EtOAc ethyl ester
THF tetrahydrofuran

### I. General Methods

### I.1 Preparation of Aryldiazonium Chlorides with Sodium Nitrite (GM 1)

To an ice bath-cooled and nitrogen-degassed solution of the aniline derivative (20.0 mmol) in hydrochloric acid (3 N, 20 ml) and water (20 ml) is added dropwise, over 10 minutes, a nitrogen-degassed solution of sodium nitrite (20.0 mmol, 1.38 g) in water (10 ml). The mixture is stirred in an ice bath for a further 20 minutes, then the clear solution can be used for further reactions. The concentration of the aryldiazonium chloride solution is 0.4 M (20.0 mmol/50 ml).

### I.2 Preparation of Aryldiazonium Tetrafluoroborates with Sodium Nitrite (GM 2)

A mixture of the particular aniline derivative (40.0 mmol), tetrafluoroboric acid (50%, 80.0 mmol, 14.0 ml) and water (15 ml) is cooled to 0-5° C. in an ice bath. A precooled solution of sodium nitrite (42.0 mmol, 2.90 g) in water (6.5 ml) is slowly added dropwise, such that the temperature always remains below 5° C. After stirring at unchanged temperature for 30 minutes, the diazonium salt is filtered off and washed with cold diethyl ether. Solvent residues are removed under reduced pressure at room temperature. The yields are between 80% and 95%. The aryldiazonium tetrafluoroborates thus obtained can be stored at below −18° C. for several weeks.

### I.3 General Method for Biaryl Synthesis (GM 3)

While stirring vigorously, a suspension of an aliquot (2.00 mmol, 5.00 ml) of the 0.4 M diazonium chloride solution from GM 1 and aqueous sodium hydroxide solution (4 N, 3 ml) is added dropwise over a period of 10-15 minutes to an aniline derivative (25.0 mmol) heated to 70° C. (or the temperature specified in the particular example). Alternatively, the suspension can also be prepared using a solution of the diazonium tetrafluoroborate (2.00 mmol, from GM 2) in a mixture of water and acetonitrile (2 ml+3 ml).

On completion of addition, the mixture is stirred for a further 10 minutes and then the reaction mixture is extracted with standard organic solvents (e.g. diethyl ether, dichloromethane or ethyl acetate) (3×75 ml). The combined organic phases are washed with saturated aqueous sodium chloride solution and dried over sodium sulfate. The solvent is removed under reduced pressure and the product obtained is dried in a vacuum. The further purification of the products is effected, according to the substance, by means of vacuum distillation, Kugelrohr distillation or column chromatography on silica gel.

### I.4 Alternative General Method for Biaryl Synthesis (GM 4)

While stirring vigorously, an aliquot (2.00 mmol, 5.00 ml) of the 0.4 M diazonium chloride solution from GM 1 is added dropwise over a period of 10-15 minutes to a suspension, heated to 70° C., of the aniline derivative (25.00 mmol) and aqueous sodium hydroxide solution (4 N, 3 ml). Alternatively, a solution of the diazonium tetrafluoroborate (2.00 ml, from GM 2) in a mixture of water and acetonitrile (2 ml+3 ml) can also be used. On completion of addition, the mixture is stirred for a further 10 minutes and then the reaction mixture is extracted with standard organic solvents (e.g. diethyl ether, dichloromethane or ethyl acetate) (3×75 ml). The combined organic phases are washed with saturated aqueous sodium chloride solution and dried over sodium sulfate. The solvent is removed under reduced pressure and the product obtained is dried in a vacuum. The further purification of the products is effected, according to the substance, by means of vacuum distillation, Kugelrohr distillation or column chromatography on silica gel.

The yields reported for the biphenyl synthesis in examples in which the diazonium salt according to GM 1 was prepared are based on the amount of aniline used, from which the diazonium salt 1 is prepared in step GM 1. In examples in which the diazonium salt was prepared according to GM 2, the yields reported for the biphenyl synthesis are based on the amount of diazonium tetrafluoroborate used.

### II. Specific Examples

### II.1 4′-Chloro-5-fluorobiphenyl-2-amine

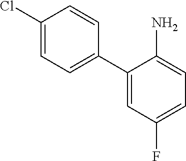 [SMILES: Nc1ccc(F)cc1-c1ccc(Cl)cc1]

To determine the reaction conditions described as GM 3, the optimization experiments which follow were conducted.

4′-Chloro-5-fluorobiphenyl-2-amine was synthesized from 4-fluoroaniline (25.0 mmol, 2.40 ml) and 4-chlorophenyldiazonium chloride (2.00 mmol, 5.00 ml of the 0.4 M aryldiazonium chloride solution prepared according to general method GM 1) in accordance with general method GM 3 and the variations of this method specified in Table 1. Diethyl ether was used for extraction, and concentration was effected under reduced pressure.

The yields reported in Table 1 in the case of performance according to GM 3 are based on the amount of 4-chloroaniline used.

| | Yield of |
|---|---|
| Reaction conditions | 4′-chloro-5-fluorobiphenyl-2-amine [%] |
| Standard conditions, see GM 3 | 47 |
| Reaction conducted at 50° C. | 46 |
| Reaction conducted at 90° C. | 51 |
| Reaction conducted at 110° C. | 48 |
| Reaction under nitrogen | 52 |
| atmosphere | |
| Reaction under argon | 48 |
| atmosphere | |
| Addition time for the diazonium | 38 |
| salt: 6 min | |
| Addition time for the diazonium | 46 |
| salt: 24 min | |
| 8N Sodium hydroxide solution | 50 |
| 4-Fluoroaniline (20.0 mmol) | 51 |
| According to GM 4 | 52 |

In the preparative experiment, 4′-chloro-5-fluorobiphenyl-2-amine was synthesized from 4-fluoroaniline (25.0 mmol, 2.40 ml) and 4-chlorophenyldiazonium chloride (2.00 mmol, 5.00 ml of the 0.4 M aryldiazonium chloride solution prepared according to general method GM 1) analogously to general method GM 3. Diethyl ether was used for extraction. The excess of 4-fluoroaniline was removed by vacuum distillation and the crude product obtained was purified by column chromatography (silica gel, hexane/EtOAc=4:1). 4′-Chloro-5-fluorobiphenyl-2-amine (0.76 mmol, 167 mg, 38%) was obtained.

R_{f} 0.4 (hexane/EtOAc=4:1) [UV]

¹H NMR (360 MHz, CDCl₃): δ=3.58 (s, 2H), 6.69 (dd, J_{HF}=4.8 Hz, J=8.6 Hz, 1H), 6.83 (dd, J=3.0 Hz, J_{HF}=9.2 Hz, 1H), 6.88 (ddd, J=3.0 Hz, J_{HF}=8.2 Hz, J=8.6 Hz, 1H), 7.38 (d, J=8.7 Hz, 2H), 7.43 (d, J=8.7 Hz, 2H).

^{13f}C NMR (90.6 MHz, CDCl₃): δ=115.2 (d, J_{CF}=22.2 Hz, CH), 116.5 (d, J_{CF}=22.6 Hz, CH), 116.6 (d, J_{CF}=7.7 Hz, CH), 127.2 (d, J_{CF}=7.1 Hz, C_{q}), 129.1 (2×CH), 130.3 (2×CH), 133.6 (C_{q}), 136.9 (d, J_{CF}=1.7 Hz, C_{q}), 139.5 (d, J_{CF}=2.1 Hz, C_{q}), 156.3 (d, J_{CF}=236.7 Hz, C_{q}).

¹⁹F NMR (339 MHz, CDCl₃): δ=−129.8.

MS (EI) m/z (%): 224 (6), 223 (29) [³⁷Cl-M⁺], 222 (18), 221 (100) [³⁵Cl-M⁺], 220 (10), 219 (20), 187 (8), 186 (45), 185 (60), 184 (13), 159 (5), 157 (7), 126 (6), 110 (10), 93 (37).

HRMS (EI) calculated for C₁₂H₉ClFN [M⁺]: 221.0407. found: 221.0407.

### II.2 4′-Chloro-5-methoxybiphenyl-2-amine and 4′-chloro-6-methoxybiphenyl-3-amine

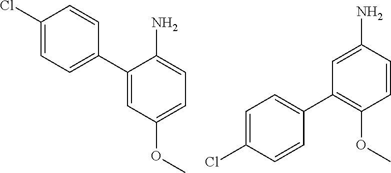

4′-Chloro-5-methoxybiphenyl-2-amine and 4′-chloro-6-methoxybiphenyl-3-amine were synthesized from p-anisidine (20.0 mmol, 2.46 g) and 4-chlorophenyldiazonium chloride (2.00 mmol, 5.00 ml of the 0.4 M aryldiazonium chloride solution prepared according to general method GM 1) analogously to general method GM 3 at 75° C. Diethyl ether was used for extraction. Excess p-anisidine was removed by vacuum distillation. The two regioisomers 4′-chloro-5-methoxybiphenyl-2-amine (0.34 mmol, 79 mg, 17%) and 4′-chloro-6-methoxybiphenyl-3-amine (0.09 mmol, 21 mg, 5%) were obtained.

### 4′-Chloro-5-methoxybiphenyl-2-amine

R_{f} 0.6 (CH₂Cl₂/EtOAc=50:1) [UV]

¹H NMR (360 MHz, CDCl₃): δ=3.76 (s, 3H), 6.69 (d, J=2.8 Hz, 1H), 6.76 (dd, J=0.6 Hz, J=8.6 Hz, 1H), 6.79 (dd, J=2.7 Hz, J=8.6 Hz, 1H), 7.41 (s, 4H).

¹³C NMR (90.6 MHz, CDCl₃): δ=55.8 (CH₃), 114.7 (CH), 115.7 (CH), 117.4 (CH), 127.9 (C_{q}), 128.9 (2×CH), 130.4 (2×CH), 133.3 (C_{q}), 136.3 (C_{q}), 137.7 (C_{q}), 153.1 (C_{q}).

### 4′-Chloro-6-methoxybiphenyl-3-amine

R_{f} 0.4 (CH₂Cl₂/EtOAc=50:1) [UV]

¹H NMR (360 MHz, CDCl₃): δ=3.71 (s, 3H), 6.66-6.69 (m, 2H), 6.81-6.84 (m, 1H), 7.36 (d, J=8.8 Hz, 2H), 7.46 (d, J=8.8 Hz, 2H).

¹³C NMR (90.6 MHz, CDCl₃): δ=56.3 (CH₃), 113.2 (CH), 115.3 (CH), 117.9 (CH), 128.1 (2×CH), 130.3 (C_{q}), 130.7 (2×CH), 132.8 (C_{q}), 136.9 (C_{q}), 140.2 (C_{q}), 149.6 (C_{q}).

### II.3 4′,5-Dichlorobiphenyl-2-amine

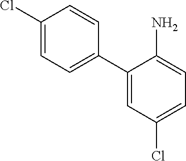 [SMILES: Nc1ccc(Cl)cc1-c1ccc(Cl)cc1]

4′,5-Dichlorobiphenyl-2-amine was synthesized from 4-chloroaniline (20.0 mmol, 2.54 g) and 4-chlorophenyldiazonium chloride (2.00 mmol, 5.00 ml of the 0.4 M aryldiazonium chloride solution prepared by general method GM 1) analogously to general method GM 3 at 80° C. Diethyl ether was used for extraction. Excess 4-chloroaniline was removed by vacuum distillation. The crude product obtained was purified by means of column chromatography (silica gel, hexane/EtOAc=4:1), which gave 4′,5-dichlorobiphenyl-2-amine (0.74 mmol, 177 mg, 37%).

Synthesis analogously to general method GM 4 gave 4′,5-dichlorobiphenyl-2-amine in a yield of 40% (0.79 mmol, 189 mg).

R_{f} 0.5 (hexane/EtOAc=4:1) [UV]

¹H NMR (360 MHz, CDCl₃): δ=6.68 (d, J=8.5 Hz, 1H), 7.06 (d, J=2.5 Hz, 1H), 7.11 (dd, J=2.5 Hz, J=8.5 Hz, 1H), 7.36 (d, J=8.7 Hz, 2H), 7.42 (d, J=8.7 Hz, 2H).

¹³C NMR (90.6 MHz, CDCl₃): δ=116.9 (CH), 123.4 (C_{q}), 128.5 (CH), 129.2 (2×CH), 129.8 (CH), 130.3 (2×CH), 131.5 (C_{q}), 133.7 (C_{q}), 136.7 (C_{q}), 141.9 (C_{q}).

MS (EI) m/z (%): 241 (10) [³⁷Cl₂-M⁺], 240 (11), 239 (29) [³⁷Cl—³⁵Cl-M⁺], 238 (19), 237 (100) [³⁵Cl₂-M⁺], 203 (12), 202 (26), 201 (31), 167 (60), 166 (18), 139 (11), 100 (17).

HRMS (EI) calculated for C₁₂H₉Cl₂N [M⁺]: 237.0112. found: 237.0112.

### II.4 4′,5-Difluorobiphenyl-2-amine

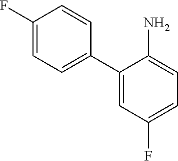 [SMILES: Nc1ccc(F)cc1-c1ccc(F)cc1]

4′,5-Difluorobiphenyl-2-amine was synthesized from 4-fluoroaniline (25.0 mmol, 2.40 ml) and 4-fluorophenyldiazonium chloride (2.00 mmol, 5.00 ml of the 0.4 M aryldiazonium chloride solution prepared by general method GM 1) analogously to general method GM 3 at 75° C. Diethyl ether was used for extraction. The excess of 4-fluoroaniline was removed by vacuum distillation and the crude product obtained was purified by column chromatography (silica gel, hexane/EtOAc=4:1). 4′,5-Difluorobiphenyl-2-amine (0.83 mmol, 170 mg, 42%) was obtained.

R_{f} 0.4 (hexane/EtOAc=4:1) [UV]

¹H NMR (360 MHz, CDCl₃): δ=6.69 (ddd, J=0.4 Hz, J_{HF}=4.9 Hz, J=8.7 Hz, 1H), 6.81-6.90 (m, 2H), 7.13 (t, J=8.7 Hz, J_{HF}=8.7 Hz, 2H), 7.40 (dd, J_{HF}=5.4 Hz, J=8.7 Hz, 2H).

¹³C NMR (90.6 MHz, CDCl₃): δ=115.0 (d, J_{CF}=22.2 Hz, CH), 115.9 (d, J_{CF}=21.4 Hz, 2×CH), 116.6 (d, J_{CF}=7.2 Hz, CH), 116.7 (d, J_{CF}=23.1 Hz, CH), 127.7 (d, J_{CF}=7.2 Hz, C_{q}), 130.6 (d, J_{CF}=8.0 Hz, 2×CH), 134.4 (dd, J_{CF}=1.7 Hz, J_{CF}=3.4 Hz, C_{q}), 139.5 (d, J_{CF}=2.3 Hz, C_{q}), 156.3 (d, J_{CF}=236.6 Hz, C_{q}), 162.2 (d, J_{CF}=247.1 Hz, C_{q}).

¹⁹F NMR (235 MHz, CDCl₃): δ=−114.0, −126.5.

MS (EI) m/z (%): 206 (13), 205 (97) [M⁺], 204 (47), 203 (56), 202 (10), 187 (10), 185 (23), 184 (17), 85 (11), 83 (16).

HRMS (EI) calculated for C₁₂H₉F₂N [M⁺]: 205.0703. found: 205.0704.

### II.5 5-Fluorobiphenyl-2-amine

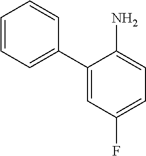 [SMILES: Nc1ccc(F)cc1-c1ccccc1]

5-Fluorobiphenyl-2-amine was synthesized from 4-fluoroaniline (25.0 mmol, 2.40 ml) and a suspension of phenyldiazonium tetrafluoroborate (2.00 mmol, 384 mg; prepared according to general method GM 2), acetonitrile (4 ml) and aqueous sodium hydroxide solution (4 N, 3 ml) analogously to general method GM 3 at 75° C. Diethyl ether was used for extraction. The excess of 4-fluoroaniline was removed by vacuum distillation and the crude product obtained was purified by column chromatography (silica gel, hexane/EtOAc=4:1). 5-Fluorobiphenyl-2-amine (0.76 mmol, 142 mg, 38%) was obtained.

R_{f} 0.3 (hexane/EtOAc=4:1) [UV]

¹H NMR (360 MHz, CDCl₃): δ=6.69 (dd, J_{HF}=4.8 Hz, J=9.4 Hz 1H), 6.84-6.90 (m, 2H), 7.33-7.39 (m, 1H), 7.42-7.48 (m, 4H).

¹³C NMR (90.6 MHz, CDCl₃): δ=114.8 (d, J_{CF}=22.2 Hz, CH), 116.4 (d, J_{CF}=7.7 Hz, CH), 116.6 (d, J_{CF}=22.5 Hz, CH), 127.6 (s, CH), 128.7 (d, J_{CF}=7.0 Hz, C_{q}), 128.9 (4×CH), 138.6 (d, J_{CF}=1.7 Hz, C_{q}), 139.6 (d, J_{CF}=2.3 Hz, C_{q}), 156.3 (d, J_{CF}=235.7 Hz, C_{q}).

¹⁹F NMR (339 MHz, CDCl₃): δ=−129.7.

MS (EI) m/z (%): 188 (13), 187 (100) [M⁺], 186 (73), 185 (31), 184 (7), 166 (3), 157 (4), 133 (4), 93 (6), 92 (5).

HRMS (EI) calculated for C₁₂H₁₀FN [M⁺]: 187.0797. found: 187.0797.

### II.6 3′,4′-Dichloro-5-fluorobiphenyl-2-amine

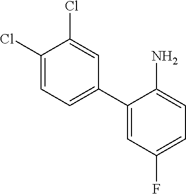 [SMILES: Nc1ccc(F)cc1-c1ccc(Cl)c(Cl)c1]

3′,4′-Dichloro-5-fluorobiphenyl-2-amine was synthesized from 4-fluoroaniline (25.0 mmol, 2.40 ml) and 3,4-dichlorophenyldiazonium tetrafluoroborate (2.00 mmol, 522 mg of the aryldiazonium tetrafluoroborate prepared according to general method GM 2, dissolved in acetonitrile (3 ml) and water (2 ml)) analogously to general method GM 3 at 70-75° C. Diethyl ether was used for extraction. The excess of 4-fluoroaniline was removed by vacuum distillation and the crude product obtained was purified by column chromatography (silica gel, hexane/EtOAc=5:1). 3′,4′-Dichloro-5-fluorobiphenyl-2-amine (0.80 mmol, 205 mg, 40%) was obtained.

R_{f} 0.3 (hexane/EtOAc=5:1) [UV]

¹H NMR (360 MHz, CDCl₃): δ=6.69 (dd, J_{HF}=4.8 Hz, J=8.8 Hz, 1H), 6.82 (dd, J=3.0 Hz, J_{HF}=9.0 Hz, 1H), 6.89 (ddd, J=3.0 Hz, J=8.1 Hz, J_{HF}=8.8 Hz, 1H), 7.29 (dd, J=2.0 Hz, J=8.2 Hz, 1H), 7.52 (d, J=8.2 Hz, 1H), 7.56 (d, J=2.1 Hz, 1H).

¹³C NMR (90.6 MHz, CDCl₃): δ=115.8 (d, J_{CF}=22.3 Hz, CH), 116.4 (d, J_{CF}=22.8 Hz, CH), 116.8 (d, J_{CF}=7.7 Hz, CH), 125.9 (d, J_{CF}=7.2 Hz, C_{q}), 128.3 (CH), 130.9 (2×CH), 131.8 (C_{q}), 133.1 (C_{q}), 138.5 (d, J_{CF}=1.7 Hz, C_{q}), 139.5 (d, J_{CF}=2.1 Hz, C_{q}), 156.3 (d, J_{CF}=237.2 Hz, C_{q}).

¹⁹F NMR (339 MHz, CDCl₃): δ=−129.0.

MS (EI) m/z (%): 259 (7) [³⁷Cl₂-M⁺], 258 (6), 257 (44) [³⁷Cl—³⁵Cl-M⁺], 256 (14), 255 (100) [³⁵Cl₂-M⁺], 220 (17), 219 (21), 186 (13), 185 (66), 184 (11), 92 (21).

HRMS (EI) calculated for C₁₂H₈Cl₂FN [M⁺]: 255.0018. found: 255.0018.

### II.7 5-Bromo-4′-chlorobiphenyl-2-amine

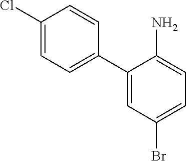 [SMILES: Nc1ccc(Br)cc1-c1ccc(Cl)cc1]

5-Bromo-4′-chlorobiphenyl-2-amine was synthesized from 4-bromoaniline (20.0 mmol, 3.44 g) and 4-chlorophenyldiazonium chloride (2.00 mmol, 5.00 ml of the 0.4 M aryldiazonium chloride solution prepared by general method GM 1) analogously to general method GM 3 at 80° C. Diethyl ether was used for extraction. Excess 4-bromoaniline was removed by vacuum distillation. The crude product obtained was purified by means of column chromatography (silica gel, hexane/EtOAc=6:1->4:1), which gave 5-bromo-4′-chlorobiphenyl-2-amine (0.62 mmol, 175 mg, 31%).

R_{f} 0.6 (hexane/EtOAc=4:1) [UV]

¹H NMR (600 MHz, CDCl₃): δ=6.67 (d, J=8.5 Hz, 1H), 7.21 (d, J=2.3 Hz, 1H), 7.25 (dd, J=2.3 Hz, J=8.5 Hz, 1H), 7.36 (d, J=8.6 Hz, 2H), 7.42 (d, J=8.6 Hz, 2H).

¹³C NMR (151 MHz, CDCl₃): δ=110.6 (C_{q}), 117.4 (CH), 129.2 (2×CH), 130.3 (2×CH), 130.6 (C_{q}), 131.4 (CH), 132.1 (C_{q}), 132.7 (CH), 133.7 (C_{q}), 136.4 (C_{q}).

MS (EI) m/z (%): 285 (23) [³⁷Cl—⁸¹Br-M⁺], 284 (10), 283 (100) [³⁷Cl—⁷⁹Br-M⁺; ³⁵Cl—⁸¹Br -M⁺], 282 (10), 281 (66) [³⁵Cl—⁷⁹Br-M⁺], 201 (12), 168 (10), 167 (73), 166 (19), 140 (11), 139 (12), 83 (27).

HRMS (EI) calculated for C₁₂H₉BrClN [M⁺]: 280.9607. found: 280.9606.

### II.8 4′-Chloro-5-cyanobiphenyl-2-amine

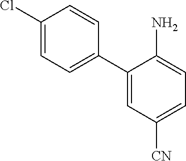 [SMILES: N#Cc1ccc(N)c(-c2ccc(Cl)cc2)c1]

4′-Chloro-5-cyanobiphenyl-2-amine was synthesized from 4-aminobenzonitrile (20.0 mmol, 2.36 g) and 4-chlorophenyldiazonium chloride (2.00 mmol, 5.00 ml of the 0.4 M aryldiazonium chloride solution prepared by general method GM 1) analogously to general method GM 3 at 95° C. Ethyl acetate was used for extraction. Excess 4-aminobenzonitrile was removed by vacuum distillation. The crude product obtained was purified by means of column chromatography (silica gel, hexane/EtOAc=3:1→2:1), which gave 4′-chloro-5-cyanobiphenyl-2-amine (0.72 mmol, 165 mg, 36%).

R_{f} 0.4 (hexane/EtOAc=2:1) [UV]

¹H NMR (600 MHz, CDCl₃): δ=6.74 (d, J=8.4 Hz, 1H), 7.33-7.35 (m, 3H), 7.42 (dd, J=1.9 Hz, J=8.4 Hz, 1H), 7.45 (d, J=8.3 Hz, 2H).

¹³C NMR (151 MHz, CDCl₃): δ=100.7 (C_{q}), 115.2 (CH), 119.8 (C_{q}), 126.1 (C_{q}), 129.5 (2×CH), 130.2 (2×CH), 132.9 (CH), 134.2 (C_{q}), 134.3 (CH), 135.5 (C_{q}), 147.5 (C_{q}).

MS (EI) m/z (%): 230 (35) [³⁷Cl-M⁺], 229 (17), 228 (100) [³⁵Cl-M⁺], 227 (10), 194 (8), 193 (49), 192 (49), 166 (9), 164 (10), 96 (14), 82 (10).

HRMS (EI) calculated for C₁₃H₉ClN₂ [M⁺]: 228.0454. found: 228.0455.

### II.9 4′-Chloro-5-ethoxybiphenyl-2-amine and 4′-chloro-6-ethoxybiphenyl-3-amine

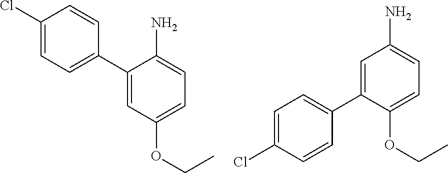

4′-Chloro-5-ethoxybiphenyl-2-amine and 4′-chloro-6-ethoxybiphenyl-3-amine were synthesized from p-phenetidine (20.0 mmol, 2.59 g) and 4-chlorophenyldiazonium chloride (2.00 mmol, 5.00 ml of the 0.4 M aryldiazonium chloride solution prepared by general method GM 1) analogously to general method GM 3 at 75° C. Diethyl ether was used for extraction. Excess p-phenetidine was removed by vacuum distillation. The crude product obtained was purified by means of column chromatography (silica gel, CH₂Cl₂/EtOAc=50:1), which gave 4′-chloro-5-ethoxybiphenyl-2-amine (0.36 mmol, 90 mg, 18%) and 4′-chloro-6-ethoxybiphenyl-3-amine (0.1 mmol, 26 mg, 5%).

### 4′-Chloro-5-ethoxybiphenyl-2-amine

R_{f} 0.6 (CH₂Cl₂/EtOAc=50:1) [UV]

¹H NMR (600 MHz, CDCl₃): δ=1.38 (t, J=7.0 Hz, 3H), 3.98 (q, J=7.0 Hz, 2H), 6. 70 (d, J=2.8 Hz, 1H), 6.73 (d, J=8.6 Hz, 1H), 6.77 (dd, J=2.8 Hz, J=8.6 Hz, 1H), 7.40 (s, 4H).

¹³C NMR (90.6 MHz, CDCl₃): δ=15.0 (CH₃), 64.2 (CH₂), 115.5 (CH), 116.6 (CH), 117.5 (CH), 127.9 (C_{q}), 128.9 (2×CH), 130.4 (2×CH), 133.3 (C_{q}), 136.1 (C_{q}), 137.8 (C_{q}), 152.2 (C_{q}).

MS (EI) m/z (%): 249 (26) [³⁷Cl-M⁺], 248 (13), 247 (75) [³⁵Cl-M⁺], 221 (15), 220 (36), 219 (40), 218 (100), 190 (15), 183 (15), 154 (17), 128 (10), 127 (10), 85 (14), 83 (18).

HRMS (EI) calculated for Cl₁₄H₁₄ClNO [M⁺]: 247.0764. found: 247.0765.

### 4′-Chloro-6-ethoxybiphenyl-3-amine

R_{f} 0.4 (CH₂Cl₂/EtOAc=50:1) [UV]

¹H NMR (360 MHz, CDCl₃): δ=1.25 (t, J=7.0 Hz, 3H), 3.88 (q, J=7.0 Hz, 2H), 6.62-6.67 (m, 2H), 6.82 (d, J=8.4 Hz, 1H), 7.34 (d, J=8.7 Hz, 2H), 7.47 (d, J=8.7 Hz, 2H).

¹³C NMR (90.6 MHz, CDCl₃): δ=14.9 (CH₃), 65.3 (CH₂), 115.4 (CH), 115.5 (CH), 117.8 (CH), 128.0 (2×CH), 128.9 (C_{q}), 130.7 (2×CH), 132.7 (C_{q}), 137.1 (C_{q}), 140.3 (C_{q}), 149.0 (C_{q}).

MS (EI) m/z (%): 249 (34) [³⁷Cl-M⁺], 248 (20), 247 (93) [³⁵Cl-M⁺], 221 (19), 220 (28), 219 (57), 218 (97), 184 (46), 183 (100), 154 (12), 128 (15), 127 (13).

HRMS (EI) calculated for C₁₄H₁₄ClNO [M⁺]: 247.0764. found: 247.0765.

### II.10 5-Fluoro-4′-methoxybiphenyl-2-amine

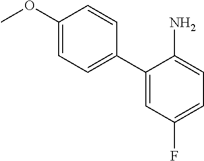 [SMILES: COc1ccc(-c2cc(F)ccc2N)cc1]

5-Fluoro-4′-methoxybiphenyl-2-amine was synthesized from 4-fluoroaniline (20.0 mmol, 1.90 ml) and 4-chlorophenyldiazonium chloride (2.00 mmol, 5.00 ml of the 0.4 M aryldiazonium chloride solution prepared by general method GM 1) analogously to general method GM 3. Diethyl ether was used for extraction. The excess of 4-fluoroaniline was removed by vacuum distillation and the crude product obtained was purified by column chromatography (silica gel, hexane/EtOAc=6:1). 5-Fluoro-4′-methoxybiphenyl-2-amine (0.26 mmol, 55 mg, 13%) was obtained.

R_{f} 0.2 (hexane/EtOAc=4:1) [UV]

¹H NMR (600 MHz, CDCl₃): δ=3.84 (s, 3H), 6.74 (dd, J_{HF}=4.8 Hz, J=9.1 Hz, 1H), 6.83-6.86 (m, 2H), 6.97 (d, J=8.7 Hz, 2H), 7.36 (d, J=8.7 Hz, 2H).

¹³C NMR (90.6 MHz, CDCl₃): δ=55.3 (CH₃), 114.3 (2×CH), 114.5 (d, J_{CF}=22.2 Hz, CH), 116.7 (d, J_{CF}=22.3 Hz, CH), 116.9 (d, J_{CF}=7.9 Hz, CH), 128.5 (d, J_{CF}=7.3 Hz, C_{q}), 130.1 (2×CH), 130.6 (d, J_{CF}=1.7 Hz, C_{q}), 138.6 (d, J_{CF}=2.2 Hz, C_{q}), 156.7 (d, J_{CF}=237.2 Hz, C_{q}), 159.1 (C_{q}).

### II.11 5-Chloro-4′-fluorobiphenyl-2-amine

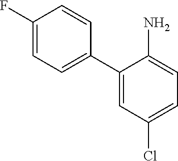 [SMILES: Nc1ccc(Cl)cc1-c1ccc(F)cc1]

5-Chloro-4′-fluorobiphenyl-2-amine was synthesized from 4-chloroaniline (20.0 mmol, 2.54 g) and 4-fluorophenyldiazonium chloride (2.00 mmol, 5.00 ml of the 0.4 M aryldiazonium chloride solution prepared by general method GM 1) analogously to general method GM 3 at 80° C. Diethyl ether was used for extraction. Excess 4-chloroaniline was removed by vacuum distillation. The crude product obtained was purified by means of column chromatography (silica gel, hexane/EtOAc=4:1), which gave 5-chloro-4′-fluorobiphenyl-2-amine (0.68 mmol, 151 mg, 34%).

In the case of synthesis according to GM 4,5-chloro-4′-fluorobiphenyl-2-amine was obtained in a yield of 35% (0.69 mmol, 153 mg).

R_{f} 0.4 (hexane/EtOAc=4:1) [UV]

¹H NMR (360 MHz, CDCl₃): δ=6.69 (d, J=8.4 Hz, 1H), 7.07 (d, J=2.2 Hz, 1H), 7.08-7.16 (m, 3H), 7.39 (dd, J_{HF}=5.3 Hz, J=8.8 Hz, 2H).

¹³C NMR (90.6 MHz, CDCl₃): δ=115.9 (d, J_{CF}=21.4 Hz, 2×CH), 116.7 (CH), 123.2 (CH), 127.9 (C_{q}), 128.3 (CH), 130.0 (d, J_{CF}=0.7 Hz, C_{q}), 130.7 (d, J_{CF}=8.1 Hz, 2×CH), 134.2 (d, J_{CF}=3.5 Hz, C_{q}), 142.2 (C_{q}), 162.3 (d, J_{CF}=247.2 Hz, C_{q}).

¹⁹F NMR (339 MHz, CDCl₃): δ=−116.9.

MS (EI) m/z (%): 223 (32) [³⁷Cl-M⁺], 222 (19), 221 (100) [³⁵Cl-M⁺], 220 (15), 219 (10), 186 (16), 185 (55), 184 (10), 92 (18).

HRMS (EI) calculated for C₁₂H₉ClFN [M⁺]: 221.0407. found: 221.0407.

### II.12 2′-Bromo-5-fluorobiphenyl-2-amine

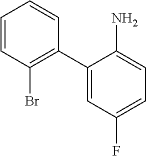 [SMILES: Nc1ccc(F)cc1-c1ccccc1Br]

2′-Bromo-5-fluorobiphenyl-2-amine was synthesized from 4-fluoroaniline (20.0 mmol, 1.90 ml) and 2-bromophenyldiazonium tetrafluoroborate (2.00 mmol, 0.54 g) analogously to general method GM 3. Diethyl ether was used for extraction. The excess of 4-fluoroaniline was removed by vacuum distillation and the crude product obtained was purified by column chromatography (silica gel, hexane/EtOAc=10:1→4:1). 2′-Bromo-5-fluorobiphenyl-2-amine (0.48 mmol, 128 mg, 24%) was obtained.

R_{f} 0.3 (hexane/EtOAc=4:1) [UV]

¹H NMR (600 MHz, CDCl₃): δ=3.29 (s, 2H), 6.70 (dd, J_{HF}=4.8 Hz, J=8.7 Hz, 1H), 6.77 (dd, J=3.0 Hz, J_{HF}=8.9 Hz, 1H), 6.92 (ddd, J=3.0 Hz, J_{HF}=8.2 Hz, J=8.7 Hz, 1H), 7.22-7.32 (m, 2H), 7.35-7.41 (m, 1H), 7.69 (dd, J=1.2 Hz, J=8.0 Hz, 1H).

¹³C NMR (90.6 MHz, CDCl₃): δ=115.6 (d, J_{CF}=22.3 Hz, CH), 116.6 (d, J_{CF}=22.7 Hz, CH), 116.5 (d, J_{CF}=7.7 Hz, CH), 123.9 (C_{q}), 127.9 (CH), 128.0 (d, J_{CF}=7.6 Hz, C_{q}), 129.6 (CH), 131.6 (CH), 133.2 (CH), 139.0 (d, J_{CF}=1.6 Hz, C_{q}), 139.7 (d, J_{CF}=2.1 Hz, C_{q}), 155.9 (d, J_{CF}=236.8 Hz, C_{q}).

### II.13 4′-Chlorobiphenyl-2-amine and 4′-chlorobiphenyl-4-amine

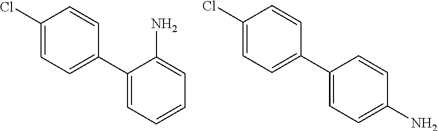 [SMILES: Nc1ccccc1-c1ccc(Cl)cc1 ; Nc1ccc(-c2ccc(Cl)cc2)cc1]

4′-Chlorobiphenyl-2-amine and 4′-chlorobiphenyl-4-amine were synthesized from aniline (20.0 mmol, 2.33 g) and 4-chlorophenyldiazonium chloride (2.00 mmol, 5.00 ml of the 0.4 M aryldiazonium chloride solution prepared by general method GM 1) analogously to general method GM 3 at 75° C. Diethyl ether was used for extraction. Excess aniline was removed by means of vacuum distillation. The two regioisomers were separated by means of column chromatography (silica gel, hexane/EtOAc=4:1). 4′-Chlorobiphenyl-2-amine (0.88 mmol, 179 mg, 44%) and 4′-chlorobiphenyl-4-amine (0.24 mmol, 49 mg, 12%) were obtained.

### 4′-Chlorobiphenyl-2-amine

R_{f} 0.6 (hexane/EtOAc=4:1) [UV]

¹H NMR (360 MHz, CDCl₃): δ=6.76 (dd, J=0.9 Hz, J=8.0 Hz, 1H), 6.82 (dt, J=1.1 Hz, J=7.47 Hz, 1H), 7.09 (dd, J=1.4 Hz, J=7.6 Hz, 1H), 7.16 (ddd, J=1.6 Hz, J=7.4 Hz, J=8.0 Hz, 1H), 7.37-7.45 (m, 4H).

¹³C NMR (90.6 MHz, CDCl₃): δ=115.7 (CH), 118.8 (CH), 126.3 (C_{q}), 128.8 (CH), 129.0 (2×CH), 130.3 (CH), 130.4 (2×CH), 133.1 (C_{q}), 137.9 (C_{q}), 143.4 (C_{q}).

MS (EI) m/z (%): 205 (29) [³⁷Cl-M⁺], 204 (10), 203 (100) [³⁵Cl-M⁺], 202 (12), 169 (17), 168 (56), 167 (37), 166 (14), 83 (29).

HRMS (EI) calculated for C₁₂H₁₀ClN [M⁺]: 203.0502. found: 203.0502.

### 4′-Chlorobiphenyl-4-amine

R_{f} 0.3 (hexane/EtOAc=4:1) [UV]

¹H NMR (600 MHz, CDCl₃): δ=6.75 (d, J=8.6 Hz, 2H), 7.35 (d, J=8.6 Hz, 2H), 7.37 (d, J=8.6 Hz, 2H), 7.45 (d, J=8.5 Hz, 2H).

¹³C NMR (151 MHz, CDCl₃): δ=115.4 (2×CH), 127.5 (2×CH), 127.8 (2×CH), 128.7 (2×CH), 130.2 (C_{q}), 132.1 (C_{q}), 139.6 (C_{q}), 146.1 (C_{q}).

MS (EI) m/z (%): 205 (32) [³⁷Cl-M⁺], 204 (18), 203 (100) [³⁵Cl-M⁺], 169 (12), 168 (9), 167 (24), 139 (10), 101 (11), 83 (21).

HRMS (EI) calculated for C₁₂H₁₀ClN [M⁺]: 203.0502. found: 203.0502.

### II.14 4′-Fluorobiphenyl-2-amine and 4′-fluorobiphenyl-4-amine

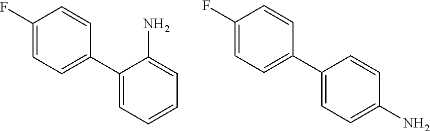 [SMILES: Nc1ccccc1-c1ccc(F)cc1 ; Nc1ccc(-c2ccc(F)cc2)cc1]

4′-Fluorobiphenyl-2-amine and 4′-fluorobiphenyl-4-amine were synthesized from aniline (25.0 mmol, 2.33 g) and 4-fluorophenyldiazonium chloride (2.00 mmol, 5.00 ml of the 0.4 M aryldiazonium chloride solution prepared by general method GM 1) analogously to general method GM 3 at 75° C. Diethyl ether was used for extraction. Excess aniline was removed by means of vacuum distillation. The two regioisomers were separated by means of column chromatography (silica gel, hexane/EtOAc=4:1). 4′-Fluorobiphenyl-2-amine (0.85 mmol, 160 mg, 43%) and 4′-fluorobiphenyl-4-amine (0.16 mmol, 30 mg, 8%) were obtained.

### 4′-Fluorobiphenyl-2-amine

R_{f} 0.5 (hexane/EtOAc=4:1) [UV]

¹H NMR (360 MHz, CDCl₃): δ=6.82 (dd, J=1.2 Hz, J=8.0 Hz, 1H), 6.86 (dt, J=1.2 Hz, J=7.5 Hz, 1H), 7.09-7.20 (m, 2H), 7.11 (t, J=8.8 Hz, J_{HF}=8.8 Hz, 2H), 7.42 (dd, J=8.8 Hz, J_{HF}=5.4 Hz, 2H).

¹³C NMR (90.6 MHz, CDCl₃): δ=115.7 (d, J_{CF}=21.4 Hz, 2×CH), 116.4 (CH), 119.6 (CH), 127.4 (CH), 128.6 (CH), 130.5 (d, J_{CF}=1.0 Hz, C_{q}), 130.8 (d, J_{CF}=8.0 Hz, 2×CH), 135.1 (d, J_{CF}=3.3 Hz, C_{q}), 142.2 (C_{q}), 162.1 (d, J_{CF}=245.3 Hz, C_{q}).

¹⁹F NMR (339 MHz, CDCl₃): δ=−118.2.

MS (EI) m/z (%): 188 (13), 187 (100) [M⁺], 186 (56), 185 (35), 184 (10), 169 (14), 168 (16), 167 (13), 123 (12), 111 (10), 95 (29), 92 (26), 83 (30), 71 (12), 57 (19).

HRMS (EI) calculated for C₁₂H₁₀FN [M⁺]: 187.0797. found: 187.0796.

### 4′-Fluorobiphenyl-4-amine

R_{f} 0.2 (hexane/EtOAc=4:1) [UV]

¹H NMR (360 MHz, CDCl₃): δ=6.75 (d, J=8.7 Hz, 2H), 7.07 (t, J_{HF}=8.8 Hz, J=8.8 Hz, 2H), 7.35 (d, J=8.7 Hz, 2H), 7.47 (dd, J_{HF}=5.3 Hz, J=8.9 Hz, 2H).

¹³C NMR (90.6 MHz, CDCl₃): δ=115.4 (d, J_{CF}=21.3 Hz, 2×CH), 115.4 (2×CH), 127.8 (d, J_{CF}=7.8 Hz, 2×CH), 127.9 (2×CH), 130.6 (C_{q}), 137.3 (d, J_{CF}=3.2 Hz, C_{q}), 145.8 (C_{q}), 161.8 (d, J_{CF}=245.0 Hz, C_{q}).

¹⁹F NMR (339 MHz, CDCl₃): δ=−120.6.

MS (EI) m/z (%): 187 (100) [M⁺], 186 (23), 170 (5), 169 (10), 159 (15), 133 (10).

HRMS (EI) calculated for C₁₂H₁₀FN [M⁺]: 187.0797. found: 187.0797.

### II.15 5-Bromo-4′-fluorobiphenyl-2-amine

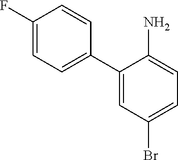 [SMILES: Nc1ccc(Br)cc1-c1ccc(F)cc1]

5-Bromo-4′-fluorobiphenyl-2-amine was synthesized from 4-bromoaniline (20.0 mmol, 2.54 g) and 4-fluorophenyldiazonium chloride (2.00 mmol, 5.00 ml of the 0.4 M aryldiazonium chloride solution prepared by general method GM 1) analogously to general method GM 3 at 80° C. Diethyl ether was used for extraction. Excess 4-bromoaniline was removed by vacuum distillation. The crude product obtained was purified by means of column chromatography (silica gel, hexane/EtOAc=6:1->4:1), which gave 5-bromo-4′-fluorobiphenyl-2-amine (0.70 mmol, 186 mg, 35%).

R_{f} 0.5 (hexane/EtOAc=4:1) [UV]

¹H NMR (600 MHz, CDCl₃): δ=6.65 (d, J=8.5 Hz, 1H), 7.13 (t, J=8.8 Hz, J_{HF}=8.8 Hz, 2H), 7.21 (d, J=2.3 Hz, 1H), 7.24 (dd, J=2.3 Hz, J=8.5 Hz, 1H), 7.38 (dd, J_{HF}=5.4 Hz, J=8.8 Hz, 2H).

¹³C NMR (90.6 MHz, CDCl₃): δ=110.6 (C_{q}), 115.9 (d, J_{CF}=21.4 Hz, 2×CH), 117.4 (CH), 128.7 (C_{q}), 130.7 (d, J_{CF}=8.0 Hz, 2×CH), 131.2 (CH), 132.8 (CH), 133.9 (d, J_{CF}=3.4 Hz, C_{q}), 142.2 (C_{q}), 162.3 (d, J_{CF}=247.3 Hz, C_{q}).

¹⁹F NMR (339 MHz, CDCl₃): δ=−117.3.

MS (EI) m/z (%): 327 (10), 268 (13), 267 (81) [⁸¹Br-M⁺], 266 (23), 265 (91) [⁷⁹Br-M⁺], 264 (12), 252 (43), 250 (23), 235 (27), 233 (16), 219 (16), 186 (27), 185 (100), 184 (23), 167 (19), 166 (16), 158 (11), 157 (21), 139 (11), 133 (13), 93 (22), 92 (37), 85 (19), 83 (29).

HRMS (EI) calculated for C₁₂H₉BrFN [M⁺]: 264.9902. found: 264.9903.

### II.16 5-Cyano-4′-fluorobiphenyl-2-amine

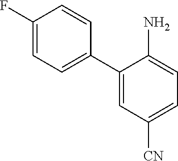 [SMILES: N#Cc1ccc(N)c(-c2ccc(F)cc2)c1]

5-Cyano-4′-fluorobiphenyl-2-amine was synthesized from 4-aminobenzonitrile (20.0 mmol, 2.36 g) and 4-fluorophenyldiazonium chloride (2.00 mmol, 5.00 ml of the 0.4 M aryldiazonium chloride solution prepared by general method GM 1) analogously to general method GM 3 at 95° C. Diethyl ether was used for extraction. Excess 4-aminobenzonitrile was removed by vacuum distillation. The crude product obtained was purified by means of column chromatography (silica gel, hexane/EtOAc=3:1→2:1), which gave 5-cyano-4′-fluorobiphenyl-2-amine (0.73 mmol, 156 mg, 37%).

R_{f} 0.3 (hexane/EtOAc=3:1) [UV]

¹H NMR (600 MHz, CDCl₃): δ=6.74 (d, J=8.4 Hz, 1H), 7.17 (t, J=8.7 Hz, J_{HF}=8.7 Hz, 2H), 7.35-7.38 (m, 3H), 7.42 (dd, J=2.0 Hz, J=8.4 Hz, 1H).

¹³C NMR (90.6 MHz, CDCl₃): δ=100.7 (C_{q}), 115.1 (CH), 116.3 (d, J_{CF}=21.5 Hz, 2×CH), 119.8 (CH), 126.4 (C_{q}), 130.7 (d, J_{CF}=8.0 Hz, 2×CH), 132.8 (CH), 133.0 (d, J_{CF}=3.6 Hz, C_{q}), 134.5 (d, J_{CF}=0.7 Hz, C_{q}), 147.7 (C_{q}), 162.5 (d, J_{CF}=247.4 Hz, C_{q}).

¹⁹F NMR (339 MHz, CDCl₃): δ=−116.5.

MS (EI) m/z (%): 212 (100) [M⁺], 211 (51), 210 (24), 193 (5), 192 (13), 184 (14), 164 (6), 157 (7), 83 (7).

HRMS (EI) calculated for C₁₃H₉FN2 [M⁺]: 212.0750. found: 212.0749.

### II.17 4′-Chloro-5-(trifluoromethyl)biphenyl-2-amine

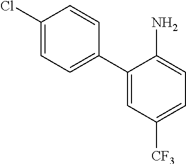 [SMILES: Nc1ccc(C(F)(F)F)cc1-c1ccc(Cl)cc1]

4′-Chloro-5-(trifluoromethyl)biphenyl-2-amine was synthesized from 4-(trifluoromethyl)aniline (20.0 mmol, 2.49 g) and 4-chlorophenyldiazonium chloride (2.00 mmol, 5.00 ml of the 0.4 M aryldiazonium chloride solution prepared by general method GM 1) analogously to general method GM 3 at 75° C. Diethyl ether was used for extraction. Excess 4-(trifluoromethyl)aniline was removed by vacuum distillation. The crude product obtained was purified by means of column chromatography (silica gel, hexane/EtOAc=6:1->4:1), which gave 4′-chloro-5-(trifluoromethyl)biphenyl-2-amine (0.73 mmol, 198 mg, 37%).

R_{f} 0.2 (hexane/EtOAc=5:1) [UV]

¹H NMR (360 MHz, CDCl₃): δ=6.79 (d, J=8.4 Hz, 1H), 7.33 (d, J=2.2 Hz, 1H), 7.38 (d, J=8.6 Hz, 2H), 7.38-7.42 (m, 1H), 7.44 (d, J=8.7 Hz, 2H).

¹³C NMR (90.6 MHz, CDCl₃): δ=115.1 (CH), 120.4 (q, J_{CF}=28.3 Hz, C_{q}), 123.2 (C_{q}), 125.9 (q, J_{CF}=3.8 Hz, CH), 126.0 (q, J_{CF}=16.5 Hz, C_{q}), 127.5 (q, J_{CF}=3.9 Hz, CH), 129.4 (2×CH), 130.4 (2×CH), 133.9 (C_{q}), 136.4 (C_{q}), 146.2 (C_{q}).

¹⁹F NMR (339 MHz, CDCl₃): δ=−64.4.

MS (EI) m/z (%): 273 (29) [³⁷Cl-M⁺], 272 (15), 271 (100) [³⁵Cl-M⁺], 236 (30), 235 (24), 216 (12), 167 (20), 85 (19), 83 (32).

HRMS (EI) calculated for C₁₃H₉ClF₃N [M⁺]: 271.0376. found: 271.0376.

### III. Amidation

### III.1 2-Chloro-N-(4′-chlorobiphenyl-2-yl)nicotinamide (Boscalid®)

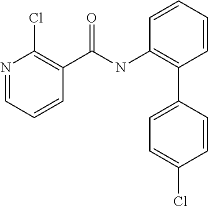 [SMILES: O=C(Nc1ccccc1-c1ccc(Cl)cc1)c1cccnc1Cl]

To a solution of 4′-chlorobiphenyl-2-amine (0.28 mmol, 58 mg) and triethylamine (1.40 mmol, 0.20 ml) in dichloromethane (4.4 ml) was slowly added, at 0° C., a solution of 2-chloronicotinyl chloride (0.41 mmol, 72 mg) in methylene chloride (0.9 ml). The mixture was allowed to thaw to room temperature for 3 h, stirred for a further hour and then heated to reflux for 2 h. The organic phase was washed with water and saturated sodium chloride solution and dried over sodium sulfate. After concentration under reduced pressure, the crude product was purified by means of column chromatography (silica gel, hexane/EtOAc=3:1) to obtain 2-chloro-N-(4′-chlorobiphenyl-2-yl)-nicotinamide (0.25 mmol; 85 mg; 87%).

R_{f} 0.4 (hexane/EtOAc=3:2) [UV].

¹H NMR (600 MHz, CDCl₃): δ=7.26-7.28 (m, 2H), 7.34 (d, J=8.4 Hz, 2H), 7.35 (m, 1H), 7.43 (d, J=8.5 Hz, 2H), 7.45-7.48 (m, 1H), 8.13 (dd, J=1.9 Hz, J=7.7 Hz, 1H), 8.14-8.17 (m, 1H), 8.41 (d, J=8.2 Hz, 1H), 8.44 (dd, J=1.9 Hz, J=4.7 Hz, 1H).

### III.2 N-(3′,4′-Dichloro-5-fluorobiphenyl-2-yl)-3-difluoromethyl-1-methyl-1H-pyrazole-4-carboxamide (Bixafen®)

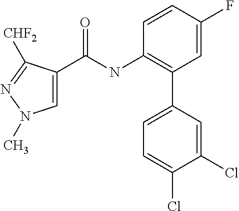 [SMILES: Cn1cc(C(=O)Nc2ccc(F)cc2-c2ccc(Cl)c(Cl)c2)c(C(F)F)n1]

A solution of 3′,4′-dichloro-5-fluorobiphenyl-2-amine (0.21 mmol, 53 mg) and 3-difluoromethyl-1-methyl-1H-pyrazole-4-carbonyl chloride (0.25 mmol, 48 mg) in THF (1 ml) was treated with triethylamine (0.41 mmol, 0.06 ml). The mixture was heated to 60° C. for 16 h. After concentration under reduced pressure, the crude product was purified by means of column chromatography (silica gel, hexane/EtOAc=3:2) to obtain N-(3′,4′-dichloro-5-fluorobiphenyl-2-yl)-3-difluoromethyl-1-methyl-1H-pyrazole-4-carboxamide (0.18 mmol, 73 mg, 85%).

R_{f} 0.1 (hexane/EtOAc=3:2) [UV].

¹H NMR (600 MHz, CDCl₃): δ=3.91 (s, 3H), 6.67 (t, J_{HF}=54.2 Hz, 1H), 6.97 (dd, J=2.9 Hz, J_{HF}=8.7 Hz, 1H), 7.12 (ddd, J=3.0 Hz, J_{HF}=8.0 Hz, J=9.0 Hz, 1H), 7.20 (dd, J=2.1 Hz, J=8.2 Hz, 1H), 7.47 (d, J=2.0 Hz, 1H), 7.50 (d, J=8.2 Hz, 1H), 7.72 (s, 1H), 7.90 (s, 1H), 8.09 (dd, J_{HF}=5.3 Hz, J=9.0 Hz, 1H).

¹³C NMR (90.6 MHz, CDCl₃): δ=39.5 (CH₃), 111.4 (t, J_{CF}=233.3, CH), 115.6 (d, J_{CF}=22.0 Hz, CH), 116.4 (C_{q}), 116.7 (d, J_{CF}=23.1 Hz, CH), 125.6 (d, J_{CF}=8.0 Hz, C_{q}), 128.4 (CH), 130.5 (d, J_{CF}=3.0 Hz, C_{q}), 130.9 (CH), 131.0 (CH), 132.6 (C_{q}), 133.1 (C_{q}), 133.9 (d, J_{CF}=7.9 Hz, C_{q}), 135.8 (C_{q}), 137.1 (d, J_{CF}=1.6 Hz, C_{q}), 142.5 (t, J_{CF}=29.0 Hz, C_{q}), 159.5 (C_{q}), 159.6 (d, J_{CF}=247.4 Hz, C_{q}).

¹⁹F NMR (339 MHz, CDCl₃): δ=−112.1, −119.7.

MS (EI) m/z (%): 417 (5) [³⁷Cl₂-M⁺], 416 (6), 415 (26) [³⁷Cl—³⁵Cl-M⁺], 414 (9), 413 (43) [³⁵Cl₂-M⁺], 219 (6), 184 (6), 160 (28), 159 (100), 139 (8), 137 (6), 83 (8), 43 (12).

HRMS (EI) calculated for C₁₈H₁₂Cl₂F₃N₃₀ [M⁺]: 413.0310. found: 413.0309.

1-27. (canceled)

28. A process for preparing a compound of the formula 3 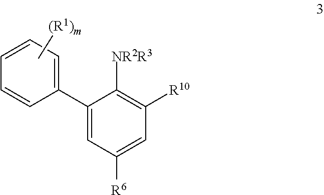
comprising reacting a compound of the formula 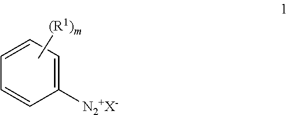
with a compound of the formula 2 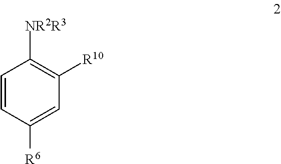
wherein
m is 0, 1, 2, 3,4 or 5;
each R¹ is independently selected from the group consisting of halogen, alkyl, haloalkyl, hydroxy, hydroxyalkyl, alkoxy, haloalkoxy, alkylthio, cycloalkyl, haloalkylthio, alkenyl, alkynyl, amino, nitro, cyano, —SO₃R⁵, —SO₂NH₂, —SO₂NHR⁴, —SO₂NR⁴R⁵, —COOR⁴, —CONHR⁴, —CONR⁴R⁵, —COR⁴, —OCOR⁴, —NR⁴R⁵, —NR⁴COR⁵, —NR⁴SO₂R⁵, alkylcarbonyl, haloalkylcarbonyl, alkenylcarbonyl, alkoxycarbonyl, haloalkoxycarbonyl, alkenyloxycarbonyl, alkylsulfonyl, haloalkylsulfonyl, alkylimino, aryl, aryloxy, arylcarbonyl, arylalkyl, heteroarylalkyl, arylalkoxycarbonyl, arylalkylimino and heteroaryl;
X⁻¹ is selected from the group consisting of halide, hydrogensulfate, sulfate, tetrafluoroborate, acetate, trifluoroacetate, hexafluorophosphate, hexafluoroantimonate, the anion of an aromatic 1,2-dicarboximide and the anion of an aromatic 1,2-disulfonimide;
R² and R³ are each independently selected from the group consisting of hydrogen, alkyl, hydroxyalkyl, aminoalkyl, cycloalkyl, haloalkyl, —(CH₂)ₙ—OR⁴, —(CH₂)ₙ—NR⁴R⁵, —(CH₂)ₙ—NR⁴COR⁵, —(CH₂)ₙ—NR⁴COOR⁵, —(CH₂)ₙ—COOR⁴, —(CH₂)ₙ—CONHR⁴, —(CH₂)ₙ—CONR⁴R⁵, —(CH₂)ₙ—SO₃R⁴, —(CH₂)ₙ—CN, arylalkyl, heteroarylalkyl, aryl and heteroaryl,
or R² and R³ together form an alkylidene radical,
or R² and R³ together with the nitrogen atom to which they are bonded form a nonaromatic 4-, 5-, 6- or 7-membered ring which may comprise 1, 2 or 3 further heteroatoms as ring members selected from O, S and N,
or R² and R¹⁰ together with the atoms to which they are bonded form a nonaromatic 4-, 5-, 6- or 7-membered ring which may comprise 1, 2 or 3 further heteroatoms as ring members selected from O, S and N,
or R³ and R¹⁰ together with the atoms to which they are bonded form a nonaromatic 4-, 5-, 6- or 7-membered ring which may comprise 1, 2 or 3 further heteroatoms as ring members selected from O, S and N;
n is in each case independently 1, 2, 3, 4, 5, 6, 7, 8, 9 or 10;
R⁴ is in each case independently selected from the group consisting of hydrogen, alkyl, cycloalkyl, haloalkyl, arylalkyl, heteroarylalkyl, aryl and heteroaryl;
R⁵ is in each case independently selected from the group consisting of hydrogen, alkyl, cycloalkyl, haloalkyl, arylalkyl, heteroarylalkyl, aryl and heteroaryl;
R⁶ is in each case independently selected from the group consisting of hydrogen, halogen, alkyl, alkenyl, alkynyl, cycloalkyl, arylalkyl, heteroarylalkyl, haloalkyl, hydroxyalkyl, alkoxyalkyl, aryloxyalkyl, heteroaryloxyalkyl, aminoalkyl, —(CH₂)ₙ—NR⁴R⁵, —COOH, —CHO, —CN, —COR⁴, alkylcarbonyl, haloalkylcarbonyl, cycloalkylcarbonyl, arylalkylcarbonyl, alkenylcarbonyl, arylcarbonyl, heteroarylcarbonyl, —COOR⁴, alkoxycarbonyl, haloalkoxycarbonyl, cycloalkoxycarbonyl, arylalkoxycarbonyl, alkenyloxycarbonyl, aryloxycarbonyl, heteroaryloxycarbonyl, —CONHR⁴, —CONR⁴R⁵, amino, nitro, —NHR⁴, —NR⁴R⁵, 1-pyrrolidino, 1-piperidino, 1-morpholino, alkylimino, cycloalkylimino, haloalkylimino, arylalkylimino, —NR⁴COR⁵, —NR⁴COOR⁵, —NR⁴SO₂R⁵, hydroxyl, alkoxy, haloalkoxy, cycloalkoxy, arylalkyloxy, aryloxy, heteroaryloxy, —OCOR⁴, alkylcarbonyloxy, haloalkylcarbonyloxy, cycloalkylcarbonyloxy, arylalkylcarbonyloxy, arylcarbonyloxy, heteroarylcarbonyloxy, —OCONR⁴R⁵, —O—(CH₂)ₙ—OR⁴, —O—(CH₂)ₙ—NR⁴R⁵, —O—(CH₂)ₙ—NR⁴COR⁵, —O—(CH₂)ₙ—NR⁴COOR⁵, —O—(CH₂)ₙ—COOR⁴, —O—(CH₂)ₙ—CONHR⁴, —O—(CH₂)ₙ—CONR⁴R⁵, —O—(CH₂)ₙ—SO₃R⁴, —O—(CH₂)ₙ—SO₂R⁴, —O—(CH₂)ₙ—CN, —SH, alkylthio, haloalkylthio, cycloalkylthio, arylalkylthio, arylthio, heteroarylthio, alkylsulfonyl, haloalkylsulfonyl, cycloalkylsulfonyl, arylalkylsulfonyl, arylsulfonyl, heteroarylsulfonyl, —SO₂NH₂, —SO₂NHR⁴, —SO₂NR⁴R⁵, —SO₃R⁵, aryl and heteroaryl;
R¹⁰ is in each case independently selected from the group consisting of hydrogen, halogen, alkyl, haloalkyl, hydroxyalkyl, cycloalkyl, arylalkyl, heteroarylalkyl, —(CH₂)_{q}—NR⁴R⁵, —(CH₂)_{q}—NR⁴COR⁵, —(CH₂)_{q}—NR⁴COOR⁵, —(CH₂)_{q}—COOR⁴, —(CH₂)_{q}—CONHR⁴, —(CH₂)_{q}—CONR⁴R⁵, —(CH₂)_{q}—SO₃R⁴, —(CH₂)_{q}—CN, aryl and heteroaryl; and
q is in each case independently 1, 2, 3, 4, or 5,
which comprises performing the reaction within the basic range.

29. A process for preparing a compound of the formula 10 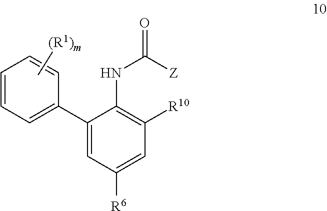
comprising
reacting a compound of the formula 1 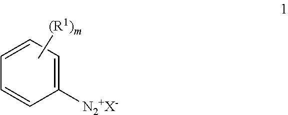
with a compound of the formula 2 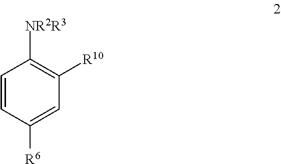
to give a compound of the formula 3 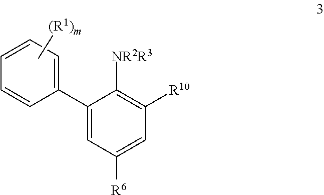
where R¹, R², R³, R⁶, R¹⁰, X⁻ and m are each as defined in claim 28; and
Z is aryl or 5- or 6-membered heteroaryl having 1, 2, 3 or 4 heteroatoms selected from the group consisting of N, O and S as ring members, where aryl and heteroaryl optionally bear 1, 2, 3 or 4 substituents selected from the group consisting of halogen, C₁-C₄-alkyl, C₁-C₄-haloalkyl, C₁-C₄-alkoxy and C₁-C₄-haloalkoxy;
which comprises performing the reaction within the basic range.

30. The process of claim 28, wherein, in a first step, a compound of the formula 1 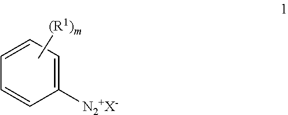
is converted within the basic range to compounds of the formula 1a, 1b or 1c, 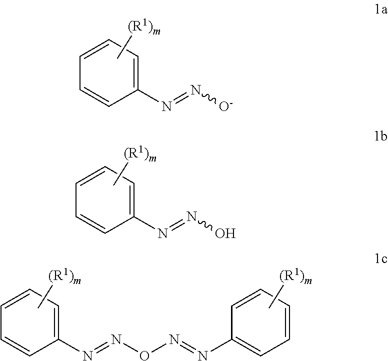
and, in a second step, the compounds 1a, 1b, or 1c is reacted within the basic range with a compound of the formula 2 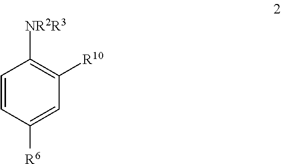
to give a compound of the formula 3 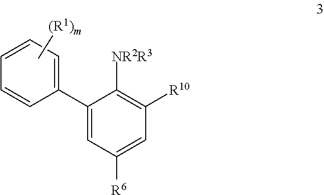
where R¹, R², R³, R⁶, R¹⁰, m and X⁻ are each as defined in claim 28.

31. The process of claim 28, wherein the reaction is performed at a pH of 9.1 or greater

32. The process of claim 28, wherein the reaction is performed in the presence of at least one solvent.

33. The process of claim 32, wherein the solvent is an aqueous solvent.

34. The process of claim 28, wherein the reaction is performed in the presence of water and of at least one base.

35. The process of claim 34, wherein the base is selected from the group consisting of alkali metal hydroxides, alkaline earth metal hydroxides, alkali metal carbonates and alkali metal phosphates, and is preferably sodium hydroxide or potassium hydroxide.

36. The process of claim 28, wherein the reaction is performed within the temperature range from 50 to 130° C.

37. The process of claim 28, wherein the compound of the formula 1 or the compound of the formula 2 or both compounds 1 and 2 are used in the reaction dispersed in an alkaline medium.

38. The process of claim 37, wherein the pH of the alkaline medium is at least 9.1.

39. The process of claim 28, wherein, in a first step, a compound of the formula 1 is reacted with a base in aqueous medium and, in a second step, the dispersion obtained is added to the compound of the formula 2.

40. The process of claim 39, wherein the pH of the dispersion obtained is at least 9.1.

41. The process of claim 39, wherein the compound 2, prior to addition of the dispersion, is brought to a temperature of 50 to 130° C.

42. The process of claim 28, wherein the compound of the formula 2 is initially charged in an alkaline medium and the compound of the formula 1 is added.

43. The process of claim 42, wherein the compound of the formula 2 is initially charged in the form of an aqueous dispersion comprising a base and the compound of the formula 1 is added to this dispersion.

44. The process of claim 42, wherein the pH of the initial charge is at least 9.1.

45. The process of claim 42, wherein the initial charge, prior to addition of the compound of the formula 1, is brought to a temperature of 50 to 130° C.

46. The process of claim 39, wherein the base is selected from the group consisting of alkali metal hydroxides, alkaline earth metal hydroxides, alkali metal carbonates and alkali metal phosphates.

47. The process of claim 46, wherein the base is sodium hydroxide or potassium hydroxide.

48. The process of claim 28, wherein R¹ is selected from the group consisting of fluorine, chlorine, bromine and methoxy.

49. The process of claim 28, wherein R², R³ and R¹⁰ are each hydrogen atoms.

50. The process of claim 28, wherein R⁶ is selected from the group consisting of hydrogen, fluorine, chlorine, bromine, CN, methoxy and ethoxy.

51. The process of claim 28, wherein m is 0, 1, 2 or 3.

52. The process of claim 28, wherein the reaction is additionally performed:
in the presence of at least one reducing agent;
under electrochemical reduction; or
under irradiation, ultrasound or radiolysis.

53. The process of claim 28, wherein the reaction is performed under protective gas.

54. The process of claim 29, wherein the preparation of a compound of the formula 10 further comprises:
N-acylation of a compound of the formula 3 in which R² and R³ are each hydrogen by reaction with a compound of the general formula 11, 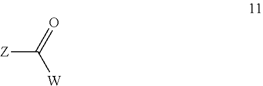
in which Z is as defined in claim 2; and
W is a leaving group
to obtain a compound of the formula 10.

55. The process of claim 54, wherein W is halogen.

56. The process of claim 29, wherein Z is 5- or 6-membered heteroaryl having 1, 2 or 3 nitrogen atoms as ring members, where the heteroaryl optionally bears 1, 2 or 3 substituents selected from the group consisting of halogen, C₁-C₄-alkyl and C₁-C₄-haloalkyl.
